(19) United States
(12) Patent Application Publication
(10) Pub. No.: US 2024/0010975 A1
(43) Pub. Date: Jan. 11, 2024

(54) GENERATION OF NEURAL PROGENITOR CELLS FROM EMBRYONIC STEM CELLS OR INDUCED PLURIPOTENT STEM CELLS

(71) Applicant: ZHEJIANG HUODE BIOENGINEERING COMPANY LIMITED, Hangzhou (CN)

(72) Inventors: Jing FAN, Hangzhou (CN); Anxin WANG, Hangzhou (CN); Fang REN, Hangzhou (CN); Qianyun LIU, Hangzhou (CN); Tan ZOU, Hangzhou (CN)

(21) Appl. No.: 18/254,549

(22) PCT Filed: May 10, 2021
(86) PCT No.: PCT/CN2021/092702

§ 371 Date: May 25, 2023

(30) Foreign Application Priority Data
Nov. 30, 2020 (WO) PCT/CN2020/132867

(51) Int. Cl. C12N 5/0797; C12N 5/0793

(52) U.S. Cl. CPC C12N 5/0623; C12N 5/0619; C12N 2506/02; C12N 2506/45; C12N 2501/727; C12N 2500/38; C12N 2501/13; C12N 2501/01; C12N 2533/52

(57) ABSTRACT

The present application provides methods, cell culture media and combinations thereof for generating neural progenitor cells (NPCs), particularly human neural progenitor cells (hNPCs), from either embryonic stem cells (ESCs) or induced pluripotent stem cells (iPSCs), wherein the NPCs are particularly suitable for pre-clinical and clinical use.

### TECHNICAL FIELD

The present application is directed to methods, cell culture media and combinations thereof for generating neural progenitor cells (NPCs), particularly human neural progenitor cells (hNPCs) from either embryonic stem cells (ESCs) or induced pluripotent stem cells (iPSCs), wherein the NPCs are particularly suitable for pre-clinical and clinical use.

### BACKGROUND

Neural progenitor cells are particularly useful in cell therapies, since they may not only self-renew and differentiate into neural cells of all types, but can also migrate and integrate into damaged parts of the central nervous system. Meanwhile, these transplanted NPCs may secrete a variety of neuroprotective and angiogenic cytokines and microRNAs, which will enhance the self-recovery process. Therefore, utilizing pluripotent NPCs to repair brain and spinal cord injuries is extremely promising in cell replacement therapy.

Since 2008 (Dimos et al. 2008), the in vitro induced differentiation of human induced pluripotent stem cells (hiPSCs) or human embryonic stem cells (hESCs) into human nerve cells and its use in research of nervous system diseases, drug screening, drug neurotoxicity testing, transplantation research and other aspects have been a hotspot in scientific research and drug development. Neural stem cells or NPCs obtained by previously existed method for inducing neural differentiation can only be further differentiated into certain subtypes of neural cells, which in general hardly have relatively mature functions either in vitro or in vivo, and also have issues like limited survival time and yield of production.

In 2016 (Xu et al. Science Translational Medicine 2016), researchers provided a novel neural differentiation method that can generate high-purity forkhead box G1 positive (FOXG1⁺) NPCs in batches from pluripotent stem cells, which can further differentiate into neural cells representative of proper cell types of the cerebral cortex, including a variety of excitatory and inhibitory neurons, and astrocytes, with mature electrophysiological functions and prolonged survival in vitro. However, this published method used reagents with animal sources, and undefined components. Thus, there is a strong need to develop a robust method to produce NPCs from pluripotent stem cells with stable quality and suitable for clinical use.

### SUMMARY OF INVENTION

The inventors of the present application have established a methodology which is capable of generating NPCs suitable for clinical and pre-clinical use from ESCs or iPSCs via directed differentiation, thus addressing above-identified needs and completing the present invention.

Therefore, in a first aspect, the present application relates to a method of generating NPCs from ESCs or iPSCs, including:
(1) culturing ESCs or iPSCs in human pluripotent stem cell medium (hPSC medium) to form embryoid bodies (EBs);
(2) culturing the EBs formed by step (1) in EB medium comprising a basal medium and supplements;
(3) culturing the EBs after step (2) on an extracellular matrix (ECM)-coated culture surface with neural induction medium (NIM) to form ROsette Neural Aggregates (RONAs), wherein the NIM comprises a basal medium and supplements;
(4) culturing the RONAs formed by step (3) to form neurospheres; and
(5) disassociating neurospheres into single cells and culturing on an ECM-coated culture surface with NPC medium to form monolayer NPCs, wherein the NPC medium comprises a basal medium and supplements;
   wherein the hPSC medium and the basal media and supplements comprised in the EB medium, the NIM, the NPC medium are clinical grade.

In a further embodiment, one or more, preferably all of the basal media and supplements in the EB medium, the NIM, and the NPC medium, are GMP grade, cGMP grade or CTS™ grade. More preferably, one or more, more preferably two, three or four, of the hPSC medium, the EB medium, the NIM, and the NPC medium are clinical grade, preferably GMP grade, cGMP grade or CTS™ grade.

In a specific embodiment, the culture surface is a plate or a flask.

In one embodiment, the hPSC medium is clinical-grade, preferably GMP grade, cGMP grade or CTS™ grade. Specifically, the hPSC medium is selected from a group consisting of NutriStem® hPSC XF Medium, CTS™ Essential 8 Medium, StemFit® Basic03, StemMACS™ iPS-Brew, Stem-Partner® ACF, TeSR™-AOF and TeSR2. In a preferred embodiment, the hPSC medium is NutriStem® hPSC XF Medium.

In one embodiment, the hPSC medium is supplemented with ROCK inhibitors. In preferred embodiments, the ROCK inhibitors are clinical grade, preferably are GMP grade, cGMP grade or CTS™ grade.

In one embodiment, the EB medium comprises
(i) a basal medium selected from a group consisting of a) to c)
   a) DMEM/F12 medium alone,
   b) DMEM/F12 in combination with Neurobasal™ Medium,
   c) KnockOut™ DMEM/F12 medium in combination with Neurobasal™ Medium; and
(ii) supplements comprising or consisting of d) or e)
   d) N-2 Supplement and GlutaMAX™-I Supplement;
   e) N-2 Supplement, GlutaMAX™-I Supplement, and B-27™ Supplement, minus vitamin A; and
(iii) optionally inhibitors.

In a further embodiment, the EB medium comprises
(i) a basal medium selected from a group consisting of a) to c)
   a) CTS™ KnockOut™ DMEM/F12 medium alone,
   b) cGMP grade DMEM/F12 in combination with CTS™ Neurobasal™ Medium,
   c) CTS™ KnockOut™ DMEM/F12 medium in combination with CTS™ Neurobasal™ Medium; and
(ii) supplements comprising or consisting of d) or e)
   d) CTS™ N-2 Supplement and CTS™ GlutaMAX™-I Supplement;
   e) CTS™ N-2 Supplement, CTS™ GlutaMAX™-I Supplement, and cGMP grade or CTS™ grade B-27™ Supplement, XenoFree, minus vitamin A; and
(iii) optionally inhibitors.

In a preferred embodiment, when the basal medium of EB medium is a), the supplements comprises or consists of e). In a specific embodiment, the supplements of the EB medium are d) or e).

In a preferred embodiment, the EB medium comprises inhibitors. Specifically, the inhibitors in the EB medium comprise or consist of a BMP inhibitor, an AMPK inhibitor and an ALK inhibitor. In a more specific embodiment, the inhibitors comprise or consist of one or more of Noggin, SB431542, LDN-193189, DMH-1 and Dorsomorphin. Preferably, the EB medium comprises SB431542 in combination with any one or more, for example one or two of Noggin, LDN-193189, DMH-1 and Dorsomorphin. In a specific embodiment, the EB medium comprises Noggin, Dorsomorphin and SB431542. In a preferred embodiment, one or more, preferably all of the inhibitors are clinical grade, preferably are GMP grade, cGMP grade or CTS™ grade.

In one embodiment, the NIM comprises
(i) a basal medium selected from a group consisting of a) to c)
   a) KnockOut™ DMEM/F12 medium alone,
   b) DMEM/F12 in combination with Neurobasal™ Medium,
   c) KnockOut™ DMEM/F12 medium in combination with Neurobasal™ Medium; and
(ii) supplements comprising or consisting of d) or e)
   d) N-2 Supplement and GlutaMAX™-I Supplement;
   e) N-2 Supplement, GlutaMAX™-I Supplement, and B-27™ Supplement, minus vitamin A.

In one embodiment, the NIM comprises
(i) a basal medium selected from a group consisting of a) to c)
   a) CTS™ KnockOut™ DMEM/F12 medium alone,
   b) cGMP grade DMEM/F12 in combination with CTS™ Neurobasal™ Medium,
   c) CTS™ KnockOut™ DMEM/F12 medium in combination with CTS™ Neurobasal™ Medium; and
(ii) supplements comprising or consisting of d) or e)
   d) CTS™ N-2 Supplement and CTS™ GlutaMAX™-I Supplement;
   e) CTS™ N-2 Supplement, CTS™ GlutaMAX™-I Supplement, and cGMP grade or CTS™ grade B-27™ Supplement, XenoFree, minus vitamin A.

In a preferred embodiment, when the basal medium of EB medium is a), the supplements comprises or consists of e). In a specific embodiment, the supplements of the EB medium are d) or e).

In step (4), RONAs are cultured to form neurospheres by using the same medium as the one being used in the step immediately before or after step (4). In one embodiment, the medium used in the step immediately before step (4), specifically NIM in step (3), is used to culture RONAs to form neurospheres. In another embodiment, the medium used in the step immediately after step (4), specifically NPC medium in step (5), is used to culture RONAs to form neurospheres.

In one embodiment, the NPC medium is Neurobasal™ Medium supplemented with GlutaMAX™-I Supplement and B-27™ Supplement, minus vitamin A. In a further embodiment, the NPC medium is CTS™ Neurobasal™ Medium supplemented with CTS™ GlutaMAX™-I Supplement, and cGMP grade or CTS™ grade B-27™ Supplement, XenoFree, minus vitamin A. In a preferred embodiment, the NPC medium further comprises brain-derived neurotrophic factor (BDNF), and/or the glial cell line-derived neurotrophic factor (GDNF), and/or L-ascorbic acid, and/or N⁶, O²′-Dibutyryl Adenosine 3′,5′ cyclic-monophosphate sodium salt (DB-cAMP). In a more specific embodiment, the BDNF is Animal-Free Recombinant BDNF or GMP grade Recombinant BDNF, and/or the GDNF is Animal-Free Recombinant GDNF or GMP grade Recombinant GDNF. In a preferred embodiment, one or more of the neurotrophic factors are GMP grade, cGMP grade or CTS™ grade. More preferably, each of the neurotrophic factors are GMP grade, cGMP grade or CTS™ grade.

In one embodiment, the neurospheres are disassociated by using a digestion enzyme in step (5). In another embodiment, the neurospheres are disassociated by mechanical means in step (5).

In one embodiment, before step (1), the ESCs or iPSCs have been maintained and expanded to 80-90% confluency, preferably in a culture surface coated with laminin. The culture surface can be a culture plate. Preferably, the laminin for coating is clinical grade.

In one embodiment, the ESCs or iPSCs in step (1) is dispersed into single cells or cell aggregates, e.g. by digestion enzyme or by mechanical means before being seeded into hPSC medium to induce formation of EBs. Preferably, a digestion enzyme is used so as to obtain more thoroughly and uniformly dispersed cells. In one embodiment, the stem cells are seeded at a density of 5,000 to 40,000 cells/well of the ultra-low attachment 96-well plate, preferably about 10,000 to 35,000 cells/well, more preferably about 20,000 to 30,000 cells/well, most preferably 30,000 cells/well in hPSC medium. In one embodiment, the stem cells are seeded as colonies the hPSC medium.

In one embodiment, the digestion enzyme used in step (1) or step (5) is CTS™ TrypLE™ Select Enzyme. In one embodiment, the digestion enzyme is clinical grade, GMP grade, cGMP grade or CTS™ grade.

In a specific embodiment, the culture of step (2) is suspension culture. In a specific embodiment, the culture of step (3) is adherent culture.

In another embodiment, more than one (e.g. two, three or more) EB medium of the present application is used in step (2). In another embodiment, more than one (e.g. two, three or more) NIM of the present application is used in step (3).

In a second aspect, the present application relates to NPCs generated by the method of the first aspect, or cells derived from the NPCs generated by the method of the first aspect. For example, the cells derived from the NPCs can be neurons, astrocyte progenitor cells, astrocytes, oligodendrocyte progenitor cells or oligodendrocytes.

In one embodiment, the majority of the NPCs generated by the method of the first aspect are FOXG1⁺ NPCs. For example, at least 50%, at least 60%, at least 70%, at least 80%, at least 90%, at least 95%, of the NPCs generated by the method are FOXG1⁺ NPCs.

In a preferred embodiment, the NPCs generated by the method of the first aspect or derived cells therefrom are suitable for pre-clinical and clinical use.

In a third aspect, the present application relates to use of the NPCs or cells derived therefrom of the second aspect in drug development or clinical applications.

In a fourth aspect, the present application relates to a method of preparing neurons, astrocyte progenitor cells, astrocytes, oligodendrocyte progenitor cells, oligodendrocytes, or a mixed population comprising any one or more of them, from the NPCs generated by the method of the first aspect. In one embodiment, the method comprises further differentiation of the NPCs. In another preferred embodiment, the method is performed with a medium of clinical grade, GMP grade, cGMP grade or CTS™ grade.

In a fifth aspect, the present application relates to neurons, astrocyte progenitor cells, astrocytes, oligodendrocyte progenitor cells or oligodendrocytes differentiated from the NPCs generated by the method of the first aspect. For example, the neurons, astrocyte progenitor cells, astrocytes, oligodendrocyte progenitor cells or oligodendrocytes are obtained by the method of the fourth aspect. Preferably, the neurons, astrocyte progenitor cells, astrocytes, oligodendrocyte progenitor cells or oligodendrocytes are suitable for pre-clinical and clinical use.

As seen from above, the EB medium and NIM can share the same essential components, including the basal medium and the supplements, which contribute to the success of the present method. Therefore, in a sixth aspect, the present application relates to use of a combination of a basal medium and supplements in EB medium and/or NIM in generating NPCs from ESCs or iPSCs, wherein the basal medium is DMEM/F12 or KnockOut™ DMEM/F12, and the supplements are N-2 Supplement and GlutaMAX™-I Supplement, and wherein the basal media and the supplements are clinical grade. Preferably, one or more of the basal media and the supplements are GMP grade, cGMP grade or CTS™ grade. In one embodiment, the basal medium further comprises clinical grade Neurobasal™ Medium, preferably CTS™ Neurobasal™ Medium. In another embodiment, the supplements further comprise clinical grade B-27™ Supplement, XenoFree, minus vitamin A, preferably cGMP grade or CTS™ grade B-27™ Supplement, XenoFree, minus vitamin A.

### BRIEF DESCRIPTION OF DRAWINGS

To facilitate a better understanding of the features and advantages of the present invention, the following detailed description and accompanying drawings thereof are provided. However, one skilled in the art will understand that they are provided with a purpose of illustration rather than limitation. The scope of the invention is controlled by the appended claims.

FIG. 1 illustrates a flow chart showing the essential steps in the generation of human neural progenitor cells (hNPCs) from human pluripotent stem cells (hPSCs), accompanied by bright-field microscopy photos showing the appearance of cell cultures at the end of each step.

FIG. 2 shows the bright-field image of clinical-grade hNPCs differentiated from hESCs (Example 1).

FIG. 3 shows the immunofluorescent staining result of the clinical-grade hNPCs obtained in Example 1 of the present invention. The term “DAPI” as used herein refers to 4′,6-diamidino-2-phenylindole.

FIG. 4 shows the flow cytometry result of the clinical-grade hNPCs obtained in Example 1 of the present invention.

FIGS. 5A-D show characterization results of the clinical-grade human neural cells after in vitro differentiation, that are obtained from the hNPCs culture in Example 1 of the present invention. FIG. 5A and FIG. 5B show the immunofluorescent staining results of cortical neuron markers (BRN2, CTIP2, TBR1 and SATB2; 5A) and synaptic protein markers (Synapsin and PSD95; 5B) after 2 months of in vitro differentiation of the clinical-grade hNPCs that are obtained from the expansion culture in Example 1 of the present invention; FIG. 5C shows the results of electrophysiological activities of the clinical-grade human neural cells at day 6 and day 13 of in vitro differentiation; and FIG. 5D shows the immunofluorescent staining results of glia cell markers after 3 months of in vitro differentiation of the clinical-grade hNPCs that are obtained from the expansion culture in Example 1 of the present invention, wherein cell nuclei are marked by DAPI, astrocytes are marked by GFAP, and oligodendrocyte progenitor cells are marked by OLIG2.

FIG. 6 is the growth curve for the clinical-grade hNPCs obtained from the expansion culture in Example 1 of the present invention.

FIG. 7 is a bright-field image of clinical-grade hNPCs differentiated from hiPSCs provided by Example 4 of the present invention.

FIG. 8 is an immunofluorescent staining result of hNPCs that are obtained from the expansion culture in Example 4 of the present invention.

FIG. 9 is a flow cytometry result of the hNPCs that are obtained from the expansion culture in Example 4 of the present invention.

FIGS. 10A-D show characterization results of the clinical grade human neural cells obtained from in vitro differentiation of the hNPCs in Example 4 of the present invention. FIG. 10A and FIG. 10B show the immunofluorescent staining results of cortical neuron markers (BRN2, CTIP2, TBR1 and SATB2; 10A) and synaptic protein markers (Synapsin and PSD95; 10B) after 2 months of in vitro differentiation from the clinical-grade hNPCs that are obtained from the expansion culture in Example 4 of the present invention; FIG. 10C shows the electrophysiological activities of the clinical-grade human neural cells at Day 6 and Day 14 of in vitro differentiation; and FIG. 10D shows the immunofluorescent staining results of glia cell markers after 3 months of in vitro differentiation of the clinical-grade hNPCs that are obtained from the expansion culture in Example 4 of the present invention, wherein cell nuclei are marked by DAPI, astrocytes are marked by GFAP, and oligodendrocyte progenitor cells are marked by OLIG2.

FIGS. 11A-E show the results of cell differentiating method using different combinations of media as described in Example 7 and Table 2. FIGS. 11A, 11B, 11C, 11D and 11E shows the results of combinations 1, 4, 5, 8 and 10, respectively.

FIG. 12 shows the brightfield image of EBs formed by the method of Example 8.

FIG. 13 is an immunofluorescent staining result of hNPCs that are obtained from the expansion culture in Example 9 of the present invention.

FIGS. 14A-D show characterization results of the clinical grade human neural cells obtained from in vitro differentiation of the hNPCs in Example 9 of the present invention. FIG. 14A and FIG. 14B show the immunofluorescent staining results of cortical neuron markers (BRN2, CTIP2, TBR1 and SATB2; 14A) and synaptic protein markers (Synapsin and PSD95; 14B) after 2 months of in vitro differentiation from the clinical-grade hNPCs that are obtained from the expansion culture in Example 9 of the present invention; FIG. 14C shows the electrophysiological activities of the clinical-grade human neural cells at Day 7 and Day 14 of in vitro differentiation; and FIG. 14D shows the immunofluorescent staining results of glia cell markers after 3 months of in vitro differentiation of the clinical-grade hNPCs that are obtained from the expansion culture in Example 9 of the present invention, wherein cell nuclei are marked by DAPI, neurons are marked by Map2, astrocytes are marked by GFAP, and oligodendrocyte progenitor cells are marked by OLIG2.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless specifically defined elsewhere in the present disclosure, all other technical and scientific terms used herein have the meaning commonly understood by one of ordinary skill in the art to which this invention belongs.

As used herein, including in the appended claims, the singular forms of words such as “a”, “an”, and “the”, include their corresponding plural references unless the context clearly dictates otherwise.

The term “or” is used to mean, and is used interchangeably with, the term “and/of” unless the context clearly dictates otherwise.

In the context of the present disclosure, unless being otherwise indicated, the wording “comprise”, and variations thereof such as “comprises” and “comprising” will be understood to imply the inclusion of a stated element, e.g. a component, a property, a step or a group thereof, but not the exclusion of any other elements, e.g. components, properties and steps. When used herein the term “comprise” or any variation thereof can be substituted with the term “contain”, “include” or sometimes “have” or equivalent variation thereof. In certain embodiments, the wording “comprise” also includes the scenario of “consisting of”.

### Clinical Grade

Without limitations to the use of other sources of agents, media, and cell-derived materials, either known or yet unknown, in the method of the present application, the term “clinical grade” with respect to the materials for generating the hNPCs, in particular the materials constituting one or more of the culture media, as well as matrix and enzymes in the present application, indicates such an agent, medium, or cell-derived material that is suitable for clinical use per se, and/or that allows directed differentiation of stem cells, in particular embryonic stem cells or induced pluripotent stem cells so as to generate safe and stable cells, especially hNPCs suitable for clinical use.

More preferably, the agent, medium, or cell-derived material used for generating the hNPCs of the present application, in particular those constituting one or more of the culture media of the present application, is GMP (good manufacturing practice) grade or cGMP (current good manufacturing practice) grade, which means said agent, medium, or cell-derived material has approved GMP or cGMP quality and is generated under GMP or cGMP standards which are defined by authorities, e.g. by WHO, MOH (Ministry of Health, P. R. China), US Food and Drug Administration or European Medicines Agency. “GMP” or “cGMP” denotes (current) standards which should be followed by manufacturers to ensure that the manufacturing process of their products are properly monitored and controlled, and their products for end users have consistently high safety.

The term “CTS” or “CTS™”, stands for cell therapy systems. It is a term used by the manufacture Thermo Fisher to indicate that a certain product for cell therapy manufacturing has high quality and meets the cGMP standards.

In the context of the present application, the phrase “of clinical use” or “suitable for clinical use” with reference to the NPCs means that the NPCs at least meet the standard as required by certain regulation for clinical and pre-clinical practices, e.g. good manufacturing practices (GMP). Specifically, the NPCs as generated via differentiation by the method of the present application have properties which render them suitable for clinical use.

### Cell Types at Different Phases

The term “neural progenitor cells” or “NPCs” as used herein refers to a type of cells that gives rise to cells of the neural lineage, including, without limitation, neurons and glial cells, for example, astrocyte progenitor cells, astrocytes, oligodendrocyte progenitor cells and oligodendrocytes by differentiation. Progenitor cells are stem cell like cells but having less capability of replication and proliferation than stem cells. However, compared to fully differentiated cells, progenitor cells still possess the capability of differentiating into different cell types. Thus, progenitor cells can serve as safer seed cells for cell replacement therapy. Successful generation of certain type of cells, in particular NPCs, can be determined by observation of morphology, detection of cell type specific biomarkers and in vitro differentiation into certain types of neural cell types with specific biomarkers and electrophysiological activities. For example, NPCs generated by the method of the present application can exhibit typical morphology of neural progenitors including a relatively homogenous columnar shape and a rosette-like radial arrangement of cells.

The abbreviation “FOXG1” or “Foxg1” as used herein stands for “forkhead box G1”, which is a one of the earliest expressed transcription factors in the development of human brain by inducing the development of the telencephalon into several critical structures including the cerebrum. The cerebral cortex is derived from forebrain forkhead box G1 (FOXG1)-expressing primordium. Therefore, FOXG1 serves as a marker for progenitor cells that will ultimately differentiate into forebrain cells. In one embodiment of the present application, the NPCs generated by the method of the present application expresses FOXG1.

The term “embryonic stem cells” or “ESCs” as used herein refers to pluripotent stem cells derived from an embryo. The term “induced pluripotent stem cells” or “iPSCs” as used herein refers to pluripotent stem cells generated by reprogramming differentiated somatic cells. The method of present application can use either ESCs or iPSCs as the starting material for directed differentiation. In specific embodiments of the present application, the ESCs or iPSCs have a mammal origin, in particular human origin. The present application does not limit the source of the stem cells, including ESCs and iPSCs, as long as they meet the requirement of clinical use. Said cells can be directly obtained from a commercial source or can be prepared by the manufacturer, for example, by reprogramming somatic cells into iPSCs.

The term “embryoid body” or “EB” as used herein refers to cell aggregates that grow in a three-dimensional manner from ESCs and iPSCs. EBs can be formed in a suspension culture. However, it is difficult to obtain EBs with uniform size and shape in the traditional way of EB culture.

In the context of the present application, the term “Rosette-type neural stem cell derived Neural Aggregates”, “ROsette Neural Aggregates” or “RONAs” as used interchangeably herein refers to aggregates of neural stem cells derived from ESCs or iPSCs and self-organized in a highly compact 3D column-like neural aggregates. The rosettes formed in the method of the present application, specifically in step (3) are immunopositive for FOXG1 and Nestin. In the method of the present application, RONAs are generally generated at day 8 after initial differentiation, i.e. about 1 day after EBs are transferred to the coated culture surface (e.g. a coated plate) in step (3) of the present method.

In the context of the present application, the term “neurosphere” as used herein refers to a sphere-shaped neural fate cell aggregates isolated from RONAs, formed from suspension culture. Neuroshpere is a heterogeneous population consisted of neural cells of different types, including neural stem cells, neural progenitor cells, and some differentiated neural cells. In the method of the present application, the neurosphere is mainly consisted of NPCs, which can be dispersed or digested into single cells and seeded to a coated culturing surface, e.g. a coated cell culture plate, to form monolayer NPCs with high purity. In the method of the present application, neurospheres are generally generated at day 21 after initial differentiation, i.e. about 1 day after RONAs are cultured in the NPC medium in step (4) of the present method. In the process of culturing RONAs to form neurospheres, a medium same to the one being used in the step immediately before or after can be used. For example, NIM for culturing RONAs or NPC medium for forming monolayer NPCs can be used.

In the present disclosure, which indicate that the cells are dislodged from the culture surface (such as culture plate) or neurospheres are disassociated by taking mechanical means or applying enzymes (such as enzymes that can disassociate the cell matrix of attachment). In preferred embodiments, the aggregated cells can be dispersed or disassociated into more uniformly discrete single cells by using a digestion enzyme as compared to mechanical means. Specifically, a treatment with a digestion enzyme can be conducted in different steps of the present method to achieve different goals. For example, the digestion in step (5) of the present method dissociates the neurospheres into single cells. Additionally, digestion enzyme can also be used (a) to disassociate and suspend the stem cells before seeding the cells in step (1) of the method, and/or (b) to disassociate NPCs during passage after step (5) of the method. The digestion enzymes used in the aforesaid steps can be the same or different. In specific embodiments, the enzyme used in the method of the present application, especially in step (1), can digest the cell colonies into discrete single cells, as compared to the enzyme which only peel away the colonies from the culture surface such as a culture plate while the cells in the colony center remained attached to each other. Exemplary enzymes can be Accutase, Dispase, Versene (EDTA) or TrypLE. In preferred embodiments, the enzyme is CTS™ TrypLE™ Select Enzyme, which can be used to digest the stem cells (ESCs or iPSCs), the neurosphere and the NPCs. In another embodiment, the enzyme in step (5) for dissociating the neurosphere is CTS™ TrypLE™ Select Enzyme. Preferably, digestion enzymes or cell detachment enzyme solutions suitable for digesting or detaching the cells in the method of the present application are clinical grade, GMP grade, cGMP grade or CTS™ grade, or the digestion enzyme is suitable for preparing NPCs for clinical use. The enzyme can be used in an amount as recommended by its manufacturer.

For the culture of iPSCs/ESCs, RONAs as well as the monolayer NPCs, the culture surface such as a culture plate is coated with extracellular matrix (ECM) to provide support for attached growth of cells. ECM is the outer cell surface matrix and is mainly consisted of proteins such as collagens, elastins and laminins. ECM is widely used in culture of mammalian cells and is known to those skilled in the art. The non-limiting examples of ECM which can be used to coat the solid support or culturing surface, e.g. a culture plate, include Matrigel™, laminin, Poly-Lysine, a combination of polyornithine (PO)/fibronectin (FN)/laminin (lam), fibronectin (FN) and the like. In preferred embodiments, the culture surface such as a plate for RONA culture is coated with Matrigel™ or laminin. In more preferred embodiments, the culture surface such as a plate for RONA culture is coated with Laminin-521 available from BioLamina, specifically MX521 or CT521, that are clinical grade or GMP grade.

The differentiation or transformation of a desired type of cells can be determined by means of immunostaining of protein markers whose expressions are specific for said type of cells. Markers conventionally used in the neuronomics are well known to one skilled in the art. For example, microtubule-associated protein 2 (Map2) isoforms a, b and c are only expressed in neurons, specifically in perikarya and dendrites.

The present application also relates to a method of generating further differentiated cells, including but not limited to neurons, and glial cells such as astrocytes and oligodendrocytes, from the NPCs generated by the present method, by e.g. culturing in a differentiation medium. The condition used for differentiation including the differentiation medium can be determined by one skilled in the art depending on the type of cells intended to grow. Preferably, the differentiation medium is clinical grade, GMP grade, cGMP grade or CTS™ grade so that the cells obtained from the differentiation of NPCs are suitable for pre-clinical and clinical use.

In a specific embodiment, the NPCs as generated by the present method are cultured in a neural differentiation medium to produce neuron. The NPC medium of the present application, e.g. the CTS-NPC medium, can be used as the neural differentiation medium, which reduces the complexity of the whole process of generating neurons from stem cells by differentiation, and can give rise to neurons suitable for pre-clinical and clinical use.

### Culture Medium

The composition of the media used for each step of the present application is essential for successful differentiation of NPCs. Any medium of the present application and the components comprised in the medium should be clinical grade, preferably GMP grade, cGMP grade or CTS™ grade, or can be used to generate NPCs suitable for clinical use by differentiation.

A medium comprises a mixture of nutrients required for the growth of cells of a certain type. A medium is usually prepared by adding supplements to a basal medium. In a broad sense, supplements refer to additional components which are not present in the basal media required by the cell culture, including proteins, lipids, amino acids, vitamins, hormones, cytokines, growth factors and the like. However, in present context, by “supplement” it does not include the inhibitors separately added to the hPSC medium and EB medium, or L-ascorbic acid, DB-cAMP, the neurotrophic factors separately added to NPC medium. The basal medium and supplements comprised in any of the media used in the method of the present application are clinical grade, preferably GMP grade, cGMP grade or CTS™ grade.

In the context of the present application, the hPSC medium is the culture medium for facilitating the formation of EBs from ESCs or iPSCs in step (1) of the present method. The hPSC medium suitable for the present method can be selected from a group consisting of NutriStem® hPSC XF Medium, CTS™ Essential 8 Medium, StemFit® Basic03, StemMACS™ iPS-Brew, Stem-Partner® ACF, TeSR™-AOF and TeSR2.

In a preferred embodiment, the hPSC medium in step (1) of the present method can be supplemented with a ROCK inhibitor. ROCK inhibitor is a type of protein kinase inhibitor which inhibits rho-associated protein kinase (ROCK), a kinase of serine-threonine protein kinase family, and prevents apoptosis of cells after dissociation or thawing. By acting on the cytoskeleton, ROCK is involved in regulating the shape and movement of cells and also regulates cellular immortalization and differentiation. Exemplary ROCK inhibitors include but not limited to Y-27632. To generate NPCs suitable for clinical use by differentiation, the ROCK inhibitors of clinical grade, GMP grade, cGMP grade or CTS™ grade are used in the method of the present application. Such ROCK inhibitors can be commercially available. The ROCK inhibitors can be added into the hPSC medium at a concentration of about 10 μM.

In the context of the present application, the EB medium is the culture medium for culturing EBs and inducing neural differentiation. Specifically, the EB medium refers to the medium used in step (2) of the present method.

In a preferred embodiment, the EB medium comprises inhibitors. More specifically, the inhibitors comprise or is consisted of a BMP inhibitor, an AMPK inhibitor and an ALK inhibitor. In one embodiment, the EB medium comprises one or more inhibitors selected from a group consisting of noggin, dorsomorphin, DMH-1, LDN-193189 and SB431542. Preferably, the EB medium comprises SB431542 in combination with any one or more, e.g. one or two of Noggin, LDN-193189, DMH-1 and Dorsomorphin. More specifically, the EB medium comprises a combination of noggin, SB431542 and dorsomorphin. The concentration of noggin in the EB medium can be 25-100 ng/mL, preferably 40-60 ng/mL, most preferably 50 ng/mL. The concentration of Dorsomorphin in the EB medium can be 0.5-2 μM, preferably 0.75-1.5 μM, most preferably 1 μM. The concentration of SB431542 in the EB medium can be 5-15 μM, preferably 7-13 μM, more preferably 8-12 μM, most preferably 10 μM.

In specific embodiments, the basal medium and supplements of the EB medium can be selected from Table 1 below. In a more specific embodiment, the EB medium has a combination of basal medium and supplements as shown in Table 1.

In the context of the present application, the NIM is the culture medium for inducing formation of RONAs.

When the basal medium of the EB medium or the NIM is made by mixing two basal media, the mixing ratio of the two media is about 1:1 by volume.

In one embodiment, the NIM and the EB medium have the same composition of basal medium and supplements and only differ in that the EB medium additionally comprises inhibitors. Using NIM and EB medium with identical essential components makes the medium preparation more efficient.

In specific embodiments, the basal medium and supplements of NIM can be selected from Table 1. In a more specific embodiment, the NIM has a combination of basal medium and supplements as shown in Table 1.

In Table 1, “CTS-DMEM/F12” refers to CTS™ KnockOut™ DMEM/F12 medium (Gibco); “DMEM/F12” refers to cGMP grade DMEM/F12 (Gibco); “CTS-NB” refers to CTS™ Neurobasal™ Medium (Gibco); “CTS-B27” refers to CTS™ grade B-27™ Supplement, XenoFree, minus vitamin A (Gibco); CTS-N2 refers to CTS™ N-2 Supplement (Gibco); and “CTS-GlutaMAX” refers to CTS™ GlutaMAX™-I Supplement (Gibco); NDS refers to noggin, dorsomorphin, and SB431542. The percentage is calculated based on volume of the media.

TABLE 1
Preferred combinations of basal medium and supplements for EB medium and NIM

| | Code name | Basal medium | | | Supplements | | |
|---|---|---|---|---|---|---|---|
| | EB medium/ | CTS-DMEM/ | DMEM/ | CTS- | CTS- | CTS- | CTS- |
| | NIM | F12 | F12 | NB | B27 | N2 | GlutaMAX |
| 1 | CTS-NDM + NDS/ | 50% | | 50% | + | + | + |
| | CTS-NDM | | | | | | |
| 2 | CTS-N2M + NDS/ | 50% | | 50% | | + | + |
| | CTS-N2M | | | | | | |
| 3 | CTS-N2B27M + NDS/ | 100% | | | + | + | + |
| | CTS-N2B27M | | | | | | |
| 4 | GMP-N2M + NDS/ | | 50% | 50% | | + | + |
| | GMP-N2M | | | | | | |

In another embodiment, more than one respective medium of the present application can be used in the culturing process of EBs and RONAs. In the EB culturing process of step (2), two or more EB media of the present disclosure, such as the EB media comprising the basal medium and supplements as listed in Table 1, can be used in any order and in any combination. Similarly, two or more NIM of the present disclosure, such as the NIM comprising the basal medium and supplements as listed in Table 1, can be used in any order and in any combination. For example, a first medium is used, followed by a second medium; or a first medium is used, then a second medium is used, and change back to the first medium; or a first medium is used, followed by a second medium and a third medium; and so on. In a more specific example, in step (2) and/or step (3), CTS grade or GMP grade N2M medium is used first, followed by CTS grade NDM medium. When more than one medium is used, medium exchange can be conducted at any time point during the culture by means well known to those skilled in the art.

In the context of the present application, the NPC medium is the culture medium for NPCs. The NPC medium preferably comprises or consists of: (i) a basal medium suitable for maintaining NPCs; and (ii) supplements comprising CTS™ GlutaMAX™-J Supplement and CTS™ grade B-27™ Supplement, XenoFree, minus vitamin A. In more preferred embodiments, the NPC medium further comprises BDNF, and/or GDNF, and/or L-ascorbic acid, and/or DB-cAMP.

Neurotrophic factors including BDNF and GDNF are agents that are important for survival, growth, or differentiation of discrete neuronal populations. BDNF or GDNF comprised in the NPC medium can be provided as an animal-free product. BDNF and/or GDNF can be clinical-grade, GMP grade, cGMP grade or CTS™ grade.

Any specific product, such as a medium, a supplement, an inhibitor, a neurotrophic factor or other agents, as exemplified in the present disclosure, may be replaced by a product of the same type but under a different trademark or product name, or may be replaced by a functional equivalent. One skilled in the art would understand that substantially the same product under a different trademark or product name, as well as functional equivalents are also encompassed by the present disclosure.

### Steps of the Method

Merely for ease of reference, the method of the present application is divided into a number of steps, each including one or more of the culturing procedures and/or treatments. Steps (1) to (5) of the method are substantially defined based on the change of culture medium and/or the form of cell cultures obtained at the end of each step.

Step (1) of the method allows stem cells, particularly ESCs or iPSCs, to form EBs by culturing the stem cells in hPSC medium. In one embodiment, the stem cells are cultured in a culture vessel such as culture plate or flask, preferably an ultra-low-attachment culture plate, more preferably an ultra-low-attachment 96-well culture plate. Typically, the culture of step (1) is conducted at 37° C. The cells are cultured for about 24 to 72 hours, preferably 2 days until they are subjected to next step. At the end of step (1), the stem cells form EBs having a morphology characterized by a round and smooth surface. The hPSC medium preferably comprises a ROCK inhibitor.

Step (2) of the method allows the EBs to grow in EB medium, in which the EBs will undergo spontaneous neural differentiation. Particularly, the EBs are cultured in suspension in step (2). In one embodiment, the stem cells are cultured in a culture vessel such as a culture plate or a flask, preferably a low-attachment culture plate, more preferably a low-attachment 6-well culture plate. Typically, the culture of step (2) is conducted at 37° C. The EBs are cultured for about 100 to 140 hours, preferably 5 days until they are moved forward to next step. Preferably, the EB medium is exchanged daily with fresh medium. At the end of step (2), the EBs have a morphology characterized by having a round, smooth surface and growing bigger. Preferred EB medium further comprises inhibitors, which facilitate to induce the neural differentiation of EBs.

Step (3) of the method allows the EBs at desired culture stage to grow in NIM to induce the formation of RONAs. In one embodiment, the EBs are attached to a culture surface such as a culture plate, a flask or a petri-dish coated with clinical-grade ECM materials. Typically, the culture of step (3) is conducted at 37° C. The neural induction of step (3) usually lasts for about 5-20 days. At the end of step (3), EBs differentiate to RONAs which are clusters of rosettes, and resulting in three-dimensional columnar cellular aggregates.

Step (4) of the method allows the rosettes-like cells in RONAs to be isolated and grown in suspension in NPC medium to form neurosphere. In one embodiment, the neural aggregates are cultured in a culture vessel such as a culture plate or a flask, preferably a low-attachment culture plate, more preferably a low-attachment 6-well culture plate. For example, the amount of cells seeded into a 6-well low-attachment culture plate at the beginning of step (4) corresponds to the amount of RONAs obtained from a 6-well culture plate. Typically, the culture of step (4) is conducted at 37° C. The neural aggregates are cultured for about 12-24 hours until they are moved forward to next step. At the end of step (4), spherical neurospheres are formed with a morphology characterized by a round and smooth surface.

Step (5) of the method allows the neurospheres to form monolayer NPCs in NPC medium. In one embodiment, step (5) includes a step (5a) of disassociation of neurospheres and a step (5b) of culture. The disassociation can be conducted by enzyme treatment or other known method such as mechanical means. The step (5a) conducted by enzyme treatment aims to disassociate the cells by treating the neurospheres with digestion enzyme for 5-10 minutes. Enzyme treated neurospheres dissociates into single cells so that they can be plated in a predetermined number for culturing. The culture step (5b) is conducted in a culture surface such as a culture plate or a flask coated with clinical-grade ECM materials. The cells are seeded at a density of about 0.5-4.0×10⁶ cells/cm², preferably 0.8-3.5×10⁶ cells/cm², more preferably 1.0×10⁶ cells/cm². Typically, the culture of step (5b) is conducted at 37° C. On next day, NPCs can be observed with a confluency of 80%-100%.

After step (5), the NPCs can be subjected to maintaining culture and passaged for one or more times. Preferably, the cells are passaged every three to five days, specifically every four days. When passage the cells, the NPCs can be treated with digestion enzyme, washed and counted before they are transferred into a culture surface such as a culture plate or a flask, preferably 6-well culture plate coated with clinical-grade ECM materials. Preferably, the medium is exchanged daily or every other day by replacing e.g. half amount of the used medium with fresh medium.

Before step (1), the present method can include maintaining of the stem cells. To start step (1), the stem cells in the maintaining culture can be treated by mechanical means or with digestion enzyme, washed and counted. One skilled in the art is capable of choosing appropriate medium for the maintaining culture. When commercially available stem cells are used, maintaining culture can be conducted under conditions recommended by the manufacturer.

### Uses

The method of the present application is suitable for producing NPCs suitable for clinical and pre-clinical uses. For example, the NPCs produced by the present method can be used in drug development, disease modeling, pre-clinical and clinical studies, and also in existing therapies and new therapies under development.

The NPCs as generated by the present method can also be used as starting materials to produce one or more types of differentiated neural cells, especially differentiated neural cells suitable for pre-clinical and/or clinical use. For example, the differentiated neural cells can be neurons or glial cells such as astrocytes, or oligodendrocytes.

### EXAMPLES

### Example 1. Generation of hNPCs from hESC by Differentiation

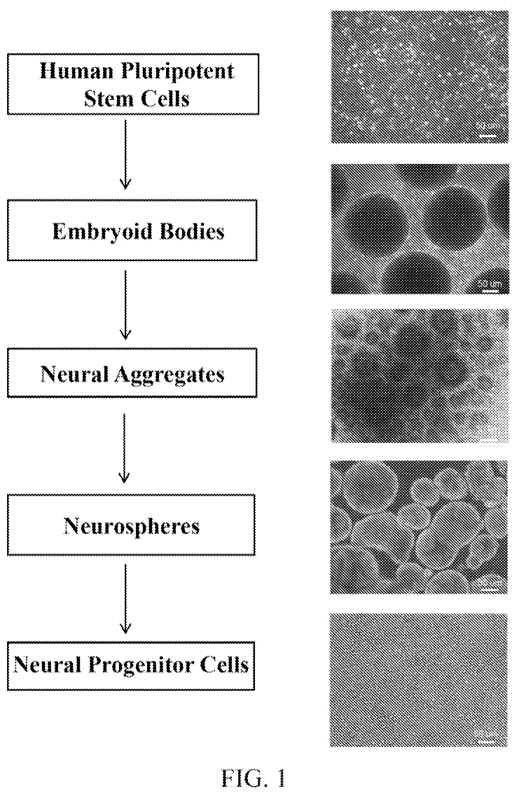

The process steps of the presently disclosed method are briefly summarized in the flow chart as shown by FIG. 1. Detailed steps and materials are described as follows.

### Preparation of Embryoid Bodies (EBs)

Human embryonic stem cell (hESC) line H1 was purchased from Shanghai Applied Cell Biotechnology Co., Ltd., (Cat. No. AC-2001002H1) and maintained in 6 well plates with NutriStem® hPSC XF Medium (Biological Industries, BI). After digesting the cells with 1 mL CTS™ TrypLE™ Select Enzyme (Gibco) per well in a 37° C. incubator with 5% CO₂ for 6 to 10 minutes, the ESCs were detached, harvested and seeded at a density of 1.65×10⁴ cells/cm² in plates coated with clinical-grade Laminin-521 (BioLamina, MX521/CT521) in 2.5 mL NutriStem® hPSC XF Medium (BI) per well.

When the confluency reached 80% to 90%, the medium was discarded, and the cells were washed with 2 mL/well CTS™ DPBS (calcium chloride- and magnesium chloride-free) (Gibco). The cells were digested with 1 mL CTS™ TrypLE™ Select Enzyme (Gibco) per well in a 37° C. incubator with 5% CO₂ for 6 to 10 minutes. When a part of the cells began to detach from the plate as observed by microscopy, the digestion was terminated by adding 3 mL of NutriStem® hPSC XF Medium (BI) to each well. After centrifuge, the cells were resuspended with NutriStem® hPSC XF Medium (BI) comprising 10 μM Rock inhibitor (CultureSure® Y-27632, Wako), and the live cells were counted.

The cells were seeded into ultra-low-attachment 96-well plates at a density of 30,000 cells per well with 200 μL NutriStem® hPSC XF Medium (BI) containing 10 μM Rock inhibitor (Wako) and cultured in an incubator at 37° C. with 5% CO₂. On the next day, it was observed that an embryoid body (EB) was formed in each well, and the EBs were the same size.

### Directed Differentiation of EBs

The cells were cultured in NutriStem® hPSC XF Medium (BI) containing 10 μM Rock inhibitor (Wako) in the ultra-low-attachment 96-well plate for 2 days (Day 0-Day 2 of directed differentiation). Then the EBs were transferred into low-attachment 6-well plates at a density of 28-32 EBs per well and the used culture medium was discarded. 2.5 mL of CTS-EB Medium was added into each well, wherein the CTS-EB Medium was prepared by mixing CTS™ KnockOut™ DMEM/F-12 Medium (Gibco) and CTS™ Neurobasal™ Medium (Gibco) at ratio of 1:1 (vol/vol) to obtain a basal medium, which is added with 50 ng/mL Noggin GMP (R&D), 1 μM Dorsomorphin (Tocris), 10 μM SB431542 (Tocris), 1 vol % of CTS™ N-2 Supplement (100×) (Gibco), and 0.5 vol % of CTS™ GlutaMAX™-I Supplement (200×) (Gibco). The EBs were cultured for 5 more days in an incubator at 37° C. with 5% CO₂, and the used medium was replaced completely with fresh CTS-EB Medium every day.

On day 7 after initial differentiation, EBs were transferred into 6 well plates coated with clinical-grade Laminin-521 (BioLamina, MX521/CT521) to allow complete attachment of EBs, and cultured with a clinical-grade serum-free neural induction medium (NIM) which promotes the expansion of the neural stem cells and NPCs. The clinical-grade serum-free NIM was prepared by mixing CTS™ KnockOut™ DMEM/F-12 Medium (Gibco) and CTS™ Neurobasal™ Medium (Gibco) at ratio of 1:1 (vol/vol) to obtain a basal medium, which was added with 1 vol % of CTS™ N-2 Supplement (100×) (Gibco), 0.5 vol % of CTS™ GlutaMAX™-I Supplement (200×) (Gibco). NIM was exchanged by replacing half of the used medium with fresh medium every other day during the first five days and was exchanged by replacing half of the used medium every day after five days. Starting from the first day of the EBs seeded in clinical-grade Laminin-521 (BioLamina, MX521/CT521) coated plates, the EBs were adhered to the plates again and gradually formed adherent cell aggregates. Later, typical neural specific rosettes were formed, indicating formation of neural stem cell aggregates. As the neural stem cells and NPCs gradually expanded, RONAs were formed in the center of the colony.

On day 21 after initial differentiation, the neural aggregates enriched with NPCs were selected and isolated under a stereomicroscope, and the selected neural aggregates were transferred into low-attachment 6-well plates containing 2.5 mL of CTS-NPC Medium in each well. The CTS-NPC Medium was prepared by adding 20 ng/mL Animal-Free Recombinant Human/Murine/Rat BDNF (PeproTech), 20 ng/mL Animal-Free Recombinant Human GDNF (PeproTech), 0.2 mM L-ascorbic acid (VC) (Sigma), 0.5 mM N⁶, O²′-Dibutyryl Adenosine 3′,5′ cyclic-monophosphate sodium salt (DB-cAMP) (Sigma), 1 vol % of CTS™ GlutaMAX™-J Supplement (100×) (Gibco) and 2 vol % of CTS™ grade B-27™ Supplement, XenoFree, minus vitamin A (50×) (Gibco) into CTS™ Neurobasal™ Medium (Gibco).

After cultured for 1 day, formation of neurospheres could be observed. The neurospheres in one well of the low-attachment 6-well plate were selected and transferred into a well of a 24-well cell culture plate. The medium was removed as carefully as possible. 0.5 mL of CTS™ TrypLE™ Select Enzyme (Gibco) was added to each well to digest the neurospheres for 5 to 10 minutes, and 1.5 mL of CTS-NPC Medium was added gently to terminate the digestion when the outer layer cells of the neurospheres became loose. The neurospheres were transferred into a 15 mL centrifuge tube and allowed for precipitation for 2 to 5 minutes. The supernatant was removed as much as possible. 1 mL of CTS-NPC Medium was added, the neurospheres were dissociated into single cells with 1 mL pipette, and the number of live cells were counted. The dissociated cells were seeded at a density of 1.0×10⁶ cells/cm² onto clinical-grade Laminin-521 (BioLamina, MX521/CT521) coated 6-well cell culture plate with 5 mL CTS-NPC Medium per well. On the next day, it could be observed that the selected hNPCs were growing in an adherent manner, with a confluency of 90% to 100%. The medium was exchanged by replacing half amount of the used medium with fresh medium every day or every other day.

The hNPCs grown in the 6-well culture plate were digested and passaged every four days. Specifically, the hNPCs were first washed once with 2 mL/well of CTS™ DPBS (Dulbecco's phosphate-buffered saline, calcium chloride- and magnesium chloride-free) (Gibco), followed by enzyme digestion with 1 mL/well of CTS™ TrypLE™ Select Enzyme (Gibco) in a 37° C. incubator with 5% CO₂ for 6 to 10 minutes. CTS™ TrypLE™ Select Enzyme solution was immediately discarded after the cells were started turning into a round shape while remaining attached to the culture plate as observed by microscopy. 3 mL of fresh CTS-NPC Medium was added into each well and pipette 8-10 times with a 1 mL pipette until almost all cells were detached. The suspension of cells was transferred into a 15 mL centrifuge tube and centrifuge at 200 g for 4 minutes. After centrifuge, the supernatant was discarded and the cell pelletes were resuspended with CTS-NPC Medium, and the number of live cells were counted. The cells were then seeded at a cell density of 1.0×10⁶ cells/cm² into clinical-grade Laminin-521 (BioLamina, MX521/CT521) coated 6-well cell culture plate with 5 mL CTS-NPC Medium in each well. On the next day, it could be observed that hNPCs were growing in an adherent manner, with a confluency of 90% to 100%. The medium was exchanged by replacing half amount of the used medium with fresh medium every day or every other day. After passaging the hNPCs for four times, the P5 hNPCs were cryopreserved or ready for direct use.

### Example 2. Characterization of the hNPCs Generated from hESC by Differentiation

In order to confirm the successful generation of hNPCs by the differentiation process as shown in Example 1, the obtained cells were subjected to verification based on observation of morphology and detection of cell specific bio-markers.

### Cell Morphology

FIG. 2 is a bright-field image of hNPCs generated by directed differentiation of hESCs following method of Example 1 of the present invention. It can be seen from FIG. 2 that the hNPCs obtained by the method provided in the present invention grew adherently and had a typical hNPC morphology, which is a relative homogenous columnar morphology and features rosette-like radial arrangements of cells.

### Immunofluorescent Staining Assay

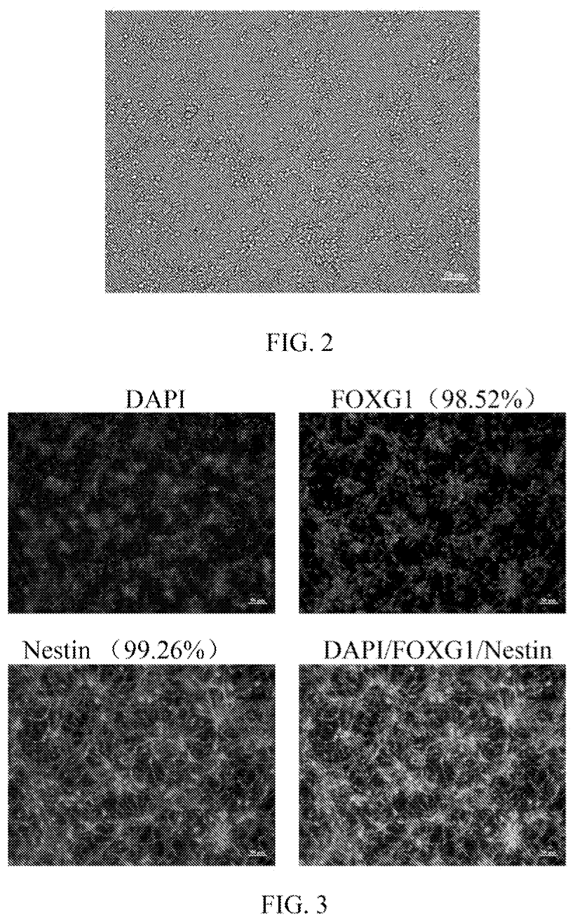

Immunofluorescent staining assay was also performed on the hNPCs obtained by the method as described in Example 1 of the present invention. As shown by FIG. 3, most of the generated cells showed expressions of both FOXG1 and Nestin, which indicated successful generation of a bulk of hNPCs with high purity by differentiation. Specifically, among the population of generated cells, a proportion of FOXG1⁺ cells as high as 98.52%, and a proportion of Nestin⁺ cells as high as 99.26% were achieved (FIG. 3).

### Flow Cytometry

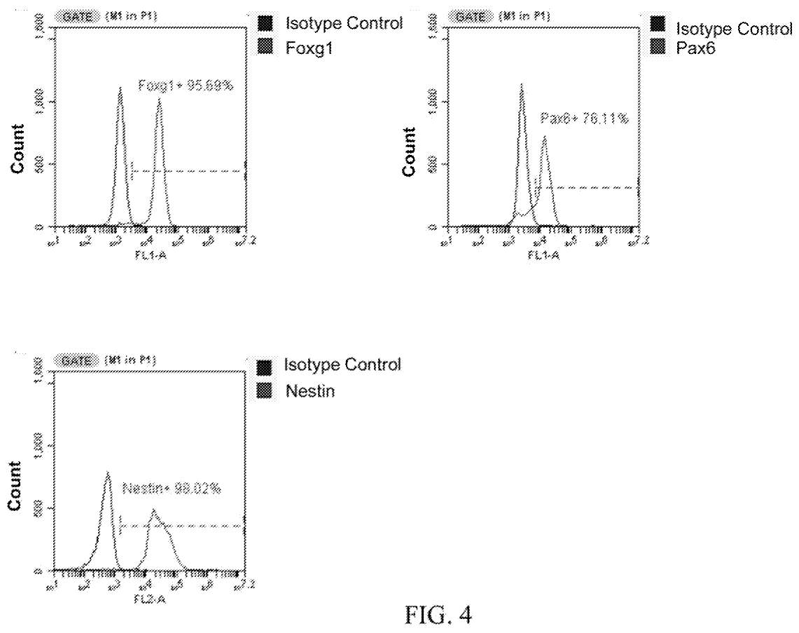

The cells generated in Example 1 were also subjected to flow cytometry which identified the hNPCs based on the expressions of FOXG1, PAX6 and Nestin. Similar results were obtained as above immunofluorescent staining assay. According to the results of flow cytometry, a proportion of FOXG1⁺ cells as high as 95.69%, and a proportion of PAX6⁺ cells as high as 76.11%, and a proportion of Nestin⁺ cells as high as 98.02% were achieved (FIG. 4).

### Example 3. Differentiation of the hNPCs Generated from hESC

Multiple experiments were conducted to test the capability of the hNPCs generated by differentiation in Example 1 to further differentiate into desirable cell types.

Differentiation of hNPCs into Neurons

The hNPCs grown in the 6-well culture plate were digested and seeded for neuron differentiation. CTS-Neural Differentiation Medium was the same as the CTS-NPC Medium as described in Example 1. The hNPCs were first washed once with 2 mL/well of CTS™ DPBS (Dulbecco's phosphate-buffered saline, calcium chloride- and magnesium chloride-free) (Gibco), followed by enzyme digestion with 1 mL/well of CTS™ TrypLE™ Select Enzyme (Gibco) in a 37° C. incubator with 5% CO₂ for 6 to 10 minutes. CTS™ TrypLE™ Select Enzyme solution was immediately discarded after the cells were started turning into a round shape while remaining attached to the culture plate as observed by microscopy. 3 mL of fresh CTS-Neural Differentiation Medium was added into each well and pipette 8-10 times with a 1 mL pipette until almost all cells were detached. The suspension of cells was transferred into a 15 mL centrifuge tube and centrifuged at 200 g for 4 minutes. After centrifuge, the supernatant was discarded and the cell pelletes were resuspended with CTS-Neural Differentiation Medium, and the number of live cells were counted. The cells were then plated at a cell density of 2.5×10⁴ live cells into clinical-grade Laminin-521 (BioLamina, MX521/CT521) coated 24-well culture plate with 500 L CTS-Neural Differentiation Medium in each well. On the next day, it could be observed that hNPCs were growing in an adherent manner. The medium was exchanged by replacing half amount of the used medium with fresh medium every 3-7 days.

### Immunofluorescent Staining Assay

Immunofluorescent staining assays were performed to show the in vitro differentiation of the hNPCs obtained from the expansion culture in Example 1 of the present invention.

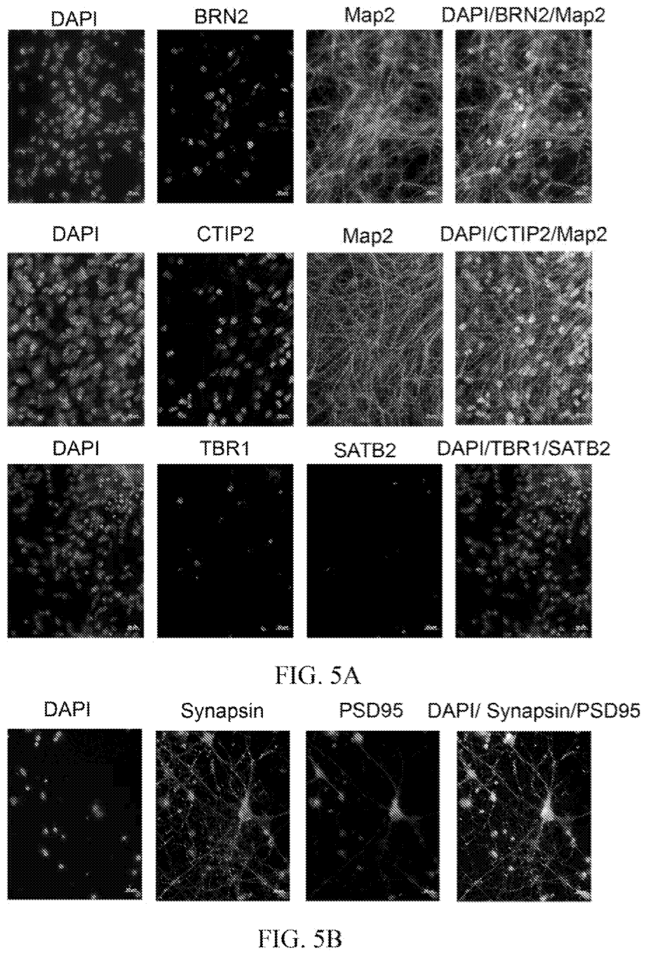

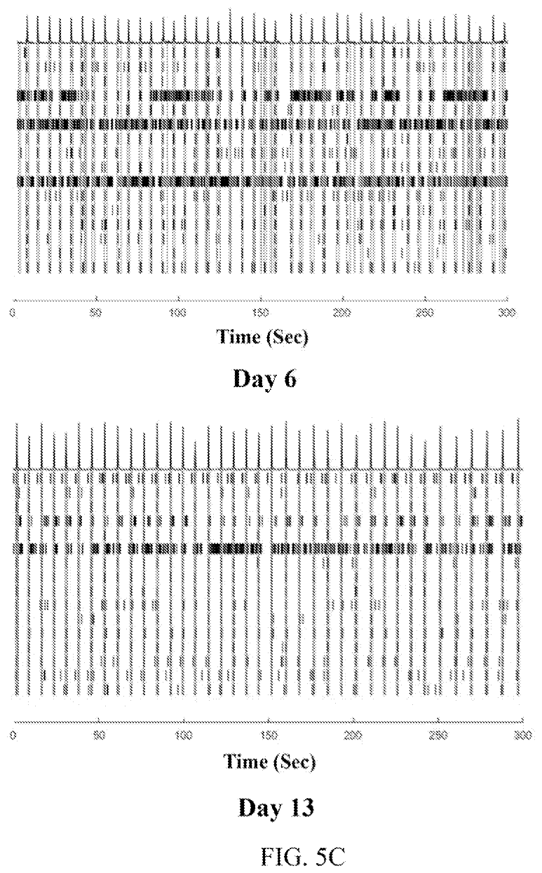

FIG. 5A shows the results of immunofluorescent staining performed with different markers specific for the six layers of human cerebral cortex on the culture of hNPCs after being differentiated in vitro for 2 months. As shown by FIG. 5A, the fluorescent signals represented expression of the markers, which were brain-2 (BRN2) and special AT-rich sequence-binding protein 2 (SATB2) for layers II to IV, chicken ovalbumin upstream promoter-transcription factor interacting protein 2 (CTIP2) for layers V and VI, T-box brain protein 1 (TBR1) for layers I, V, and VI, and Map2 (Microtubule-associated protein 2) for neurons. The results of the immunostaining demonstrated that the hNPCs obtained by the method of Example 1 had successfully differentiated into neural cells of all six layers of human cerebral cortex.

FIG. 5B shows the results of immunostaining for synapsin and postsynaptic density protein 95 (PSD95), which are presynaptic marker and postsynaptic marker, respectively. As shown by FIG. 5B, the signals representing synaptic proteins distribute as discrete points, indicating maturation of neurons. As shown by FIG. 5C, electrophysiological activities could be detected within one week (Day 6) of in vitro differentiation. Moreover, after extension of the culture time (Day 13), spike firing gradually intensified, and more regular spontaneous firing, single bursts and network bursts were generated.

After 3 months of in vitro differentiation of hNPCs obtained by the method as described in Example 1, immunofluorescent staining assays were performed to detect the expression of glial fibrillary acidic protein (GFAP) and oligodendrocyte transcription factor 2 (OLIG2). As shown by FIG. 5D, the positive signals of GFAP and OLIG2 indicated that astrocytes and oligodendrocyte progenitor cells were further differentiated from the hNPCs obtained by the method of the present application.

Taken together, the results of the above-mentioned immunostaining assays demonstrated that the hNPCs generated from ESCs by the method as described in Example 1 are capable of further differentiating into neural cells of all six layers of human cerebral cortex with desirable physiological functions.

### Growth Curve

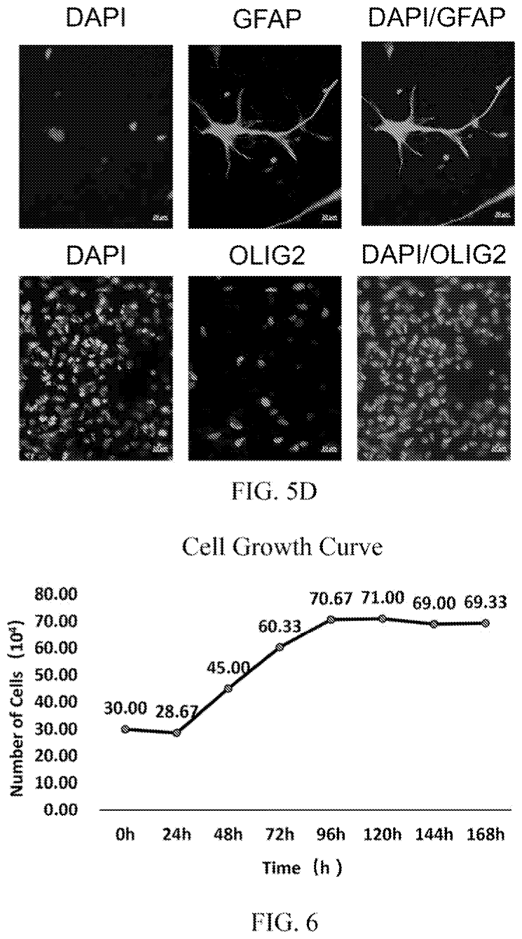

FIG. 6 is a growth curve of the hNPCs (P5) obtained in Example 1 of the present invention. The hNPCs doubling time was calculated according to a doubling time formula PDT=lg2×(T−T₀)/(lgN_{T}−lgN₀), wherein PDT is doubling time, T is the time point when the cell number is counted, T₀ is the time point when the cells are seeded, N_{T} is the cell count at the time point T, and N₀ is the seeded cell number. The hNPCs doubling time was calculated as 73 hours.

Also, it can be seen from FIG. 6 that the hNPCs cells started to proliferate at 24 h. The cell count was about two times of the seeded cell number at 72 h and a plateau was reached at 96 h. Thereafter, the cell number stayed at a relatively high level with no increase, indicating loss of proliferation. Cells at this stage still possessed potential for differentiation in vitro. Therefore, passaging the cells after four days could produce the maximum yield of the NPCs of the present application.

### Example 4. Generation of hNPCs from iPSC by Differentiation

The same process as described in Example 1 was performed except starting with human induced pluripotent stem cells (iPSC) purchased from Stem Cell Bank, Chinese Academy of Sciences under Cat. No. SCSP-1301, instead of hESC line H1.

### Example 5. Characterization of the hNPCs Generated from iPSCs by Differentiation

In order to confirm the successful generation of hNPCs from iPSC by the differentiation process of Example 4, the obtained cells were subjected to verification based on observation of morphology and detection of cell specific bio-markers.

### Cell Morphology

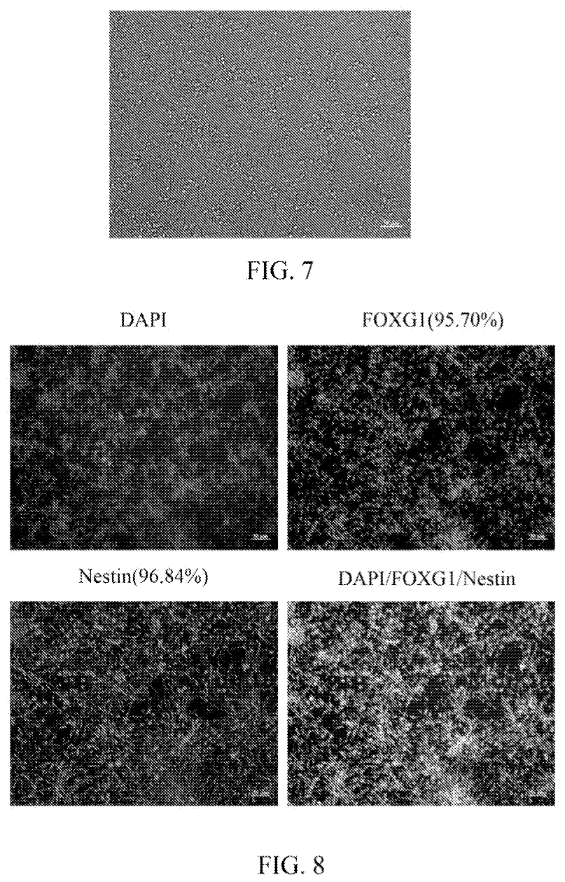

FIG. 7 is a bright-field image of hNPCs generated by directed differentiation of iPSCs following method of Example 4 of the present invention. It can be seen from FIG. 7 that the hNPCs grew adherently and had a typical hNPC morphology, which is a relatively homogenous columnar morphology and features rosette-like radial arrangements of cells.

### Immunofluorescent Staining Assay

Immunofluorescent staining assay was also performed on the hNPCs obtained by the method as described in Example 4 of the present invention. As shown by FIG. 8, most of the cells showed expressions of both FOXG1 and Nestin, two molecular markers specifically for the hNPCs of forebrain region, indicating successful generation of a bulk of hNPCs with high purity by differentiation. Specifically, among the population of generated cells, a proportion of FOXG1⁺ cells as high as 95.70%, and a proportion of Nestin⁺ cells as high as 96.84% were achieved (FIG. 8).

### Flow Cytometry

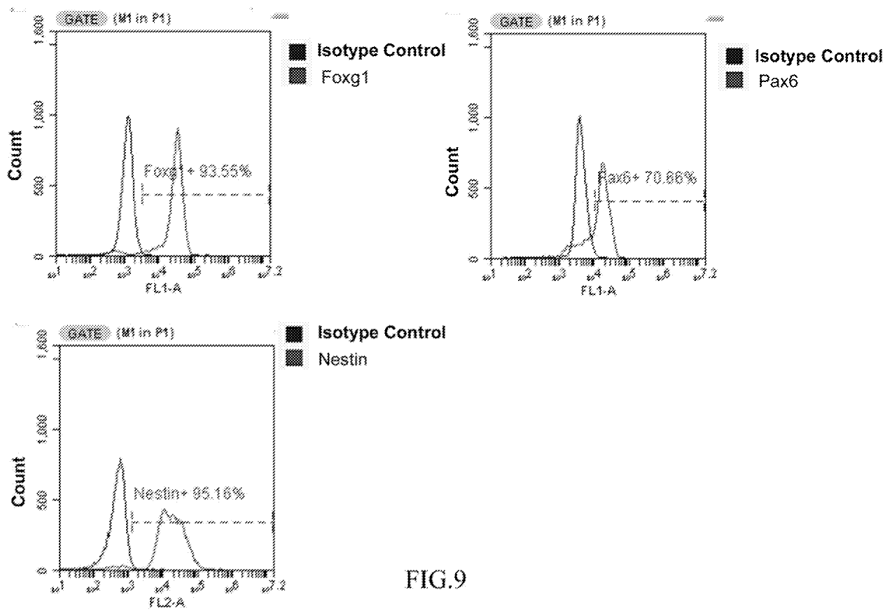

The cells generated in Example 4 were also subjected to flow cytometry assays which identified the hNPCs based on the expressions of FOXG1, PAX6 and Nestin. Similar results were obtained as above immunofluorescent staining assay. According to the results of flow cytometry, a proportion of FOXG1⁺ cells as high as 93.55%, a proportion of PAX6⁺ cells as high as 70.66%, and a proportion of Nestin⁺ cells as high as 95.16% were achieved (FIG. 9).

### Example 6. Differentiation of the hNPCs Generated from iPSCs

Multiple experiments were conducted to test the capability of the hNPCs generated by differentiation in Example 4 to further differentiate into desirable cell types, e.g. neurons.

Differentiation of hNPCs into Neurons

The differentiation of hNPCs was conducted in the same way as described in Example 3.

### Immunofluorescent Staining Assay

Immunofluorescent staining assays were performed to show the in vitro differentiation of the hNPCs obtained from the expansion culture in Example 4 of the present invention.

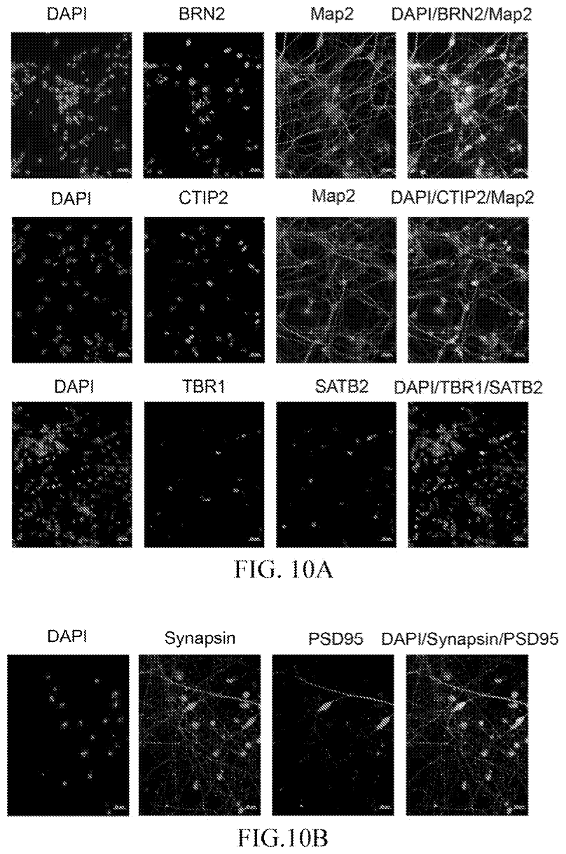

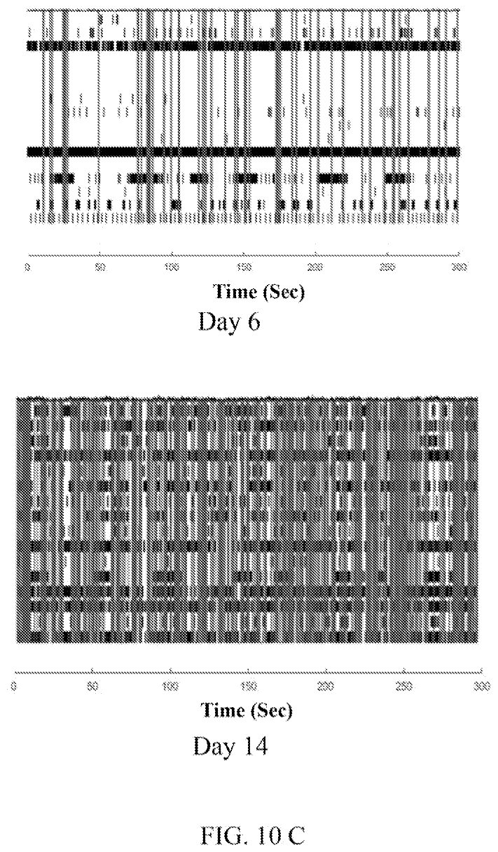

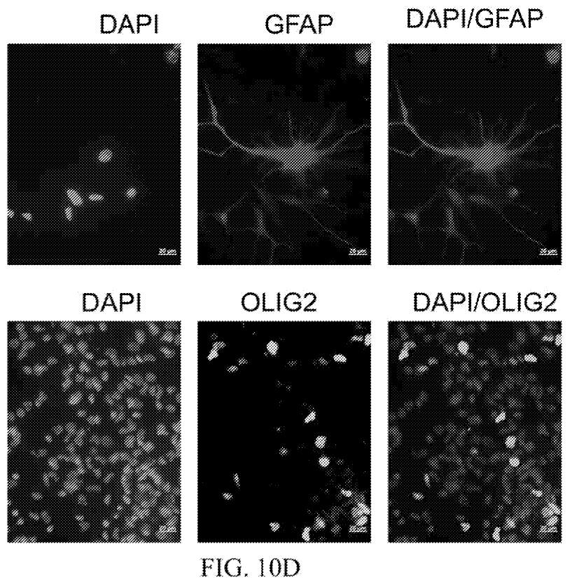

FIG. 10A shows the results of immunofluorescent staining performed after 2 months of in vitro differentiation of hNPCs, which detected the expression of the markers specific for the six layers of human cerebral cortex. As shown by FIG. 10A, the fluorescent signals represented expression of the markers, which were BRN2 and SATB2 for layers II to IV, CTIP2 for layers V and VI, TBR1 for layers I, V, and VI, and Map2 for neurons. The results of the immunostaining demonstrated that the hNPCs obtained by the method of Example 4 had successfully differentiated into nerve cells of all six layers of human cerebral cortex.

FIG. 10B shows the results of immunofluorescent staining for Synapsin and PSD95, indicating the expression of these two markers which are presynaptic marker and postsynaptic marker, respectively. As shown by FIG. 10B, the signals representing expression of synaptic protein were distributed as discrete points, indicating maturation of neurons after differentiation. As shown by the FIG. 10C, electrophysiological activities could be detected on Day 6 of in vitro differentiation. Moreover, as the culture time was extended, spike firing gradually intensified, and more regular spontaneous firing and network bursts were observed.

After 3 months of in vitro differentiation of hNPCs obtained by the method as described in Example 4, immunofluorescent staining assays were performed to detect the expression of GFAP and OLIG2. As shown by FIG. 10D, the positive signals of GFAP and OLIG2 indicated that astrocytes and oligodendrocyte progenitor cells were further differentiated from the hNPCs obtained by the method of the present application at this time point.

Taken together, the above-mentioned immunostaining assays demonstrated that the hNPCs generated from iPSCs by the method as described in Example 4 are capable of differentiating into cells of all six layers of human cerebral cortex with desirable physiological functions.

### Example 7. Directed Differentiation with Different Culturing Media

The present inventors tested a series of different culturing media of clinical grade in the method as described in Example 1. Specifically, the composition of basal medium and supplements of the EB medium and NIM were changed in every test while the rest of the conditions remained the same. The results are summarized in Table 2 and Table 3 below.

The medium combinations 1 to 4 of Table 2 and Table 3 are identical to those listed in Table 1, while the combinations 5 to 10 are additionally tested medium compositions. The EB medium and NIM of combinations 6 to 10 are different in the composition of basal medium and supplements. “NEAA” refers to GMP grade MEM Non-Essential Amino Acids Solution; and “CTS-KOSR” refers to KnockOut™ SR XenoFree CTS™. “BI-EB” refers to NutriStem® hPSC XF Medium (Growth Factor-Free).

TABLE 2
Testing results of different EB and NIM compositions

| | Code name | Basal medium | | | | Supplements | | | | |
|---|---|---|---|---|---|---|---|---|---|---|
| | EB medium/ | CTS-DMEM/ | DMEM/ | CTS- | CTS- | CTS- | CTS- | CTS- | | |
| No. | NIM | F12 | F12 | NB | KOSR | B27 | N2 | GlutaMAX | NEAA | Rating |
| 1 | CTS-NDM + NDS/ | 50% | | 50% | | + | + | + | | EXCELLENT |
| | CTS-NDM | | | | | | | | | |
| 2 | CTS-N2M + NDS/ | 50% | | 50% | | | + | + | | EXCELLENT |
| | CTS-N2M | | | | | | | | | |
| 3 | CTS-N2B27M + | 100% | | | | + | + | + | | EXCELLENT |
| | NDS/CTS-N2B27M | | | | | | | | | |
| 4 | GMP-N2M + NDS/ | | 50% | 50% | | | + | + | | FAIRLY GOOD |
| | GMP-N2M | | | | | | | | | |
| 5 | GMP-N2B27M + | | 100% | | | + | + | + | | BAD |
| | NDS/GMP-N2B27M | | | | | | | | | |
| 6 | GMP-EBM + NDS/ | 100% | | | | | + | + | + | BAD |
| | GMP-NIM | | 100% | | | | + | + | + | |
| 7 | GMP-EBM + NDS/ | | 80% | | 20% | | | + | + | BAD |
| | GMP-NIM | | 100% | | | | + | + | + | |
| 8 | BI-EB + NDS/ | | | | | | | | | EXTREMELY |
| | CTS-NIM | 100% | | | | | + | + | + | BAD |
| 9 | CTS-NIM + NDS/ | 100% | | | | | + | + | + | EXTREMELY |
| | CTS-NIM | 100% | | | | | + | + | + | BAD |
| 10 | CTS-EBM + NDS/ | 80% | | | 20% | | | + | + | EXTREMELY |
| | CTS-NIM | 100% | | | | | + | + | + | BAD |

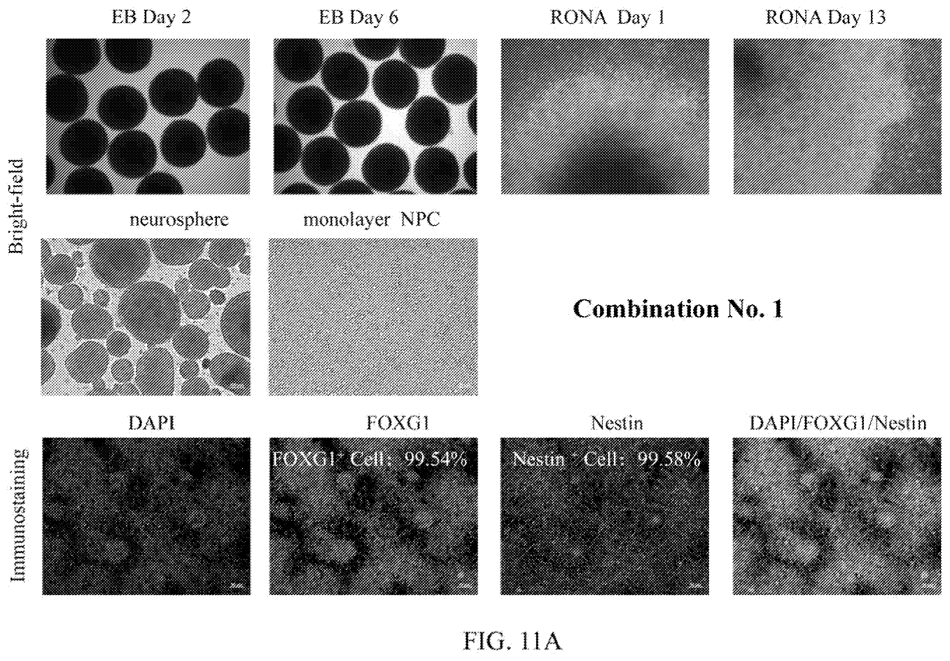

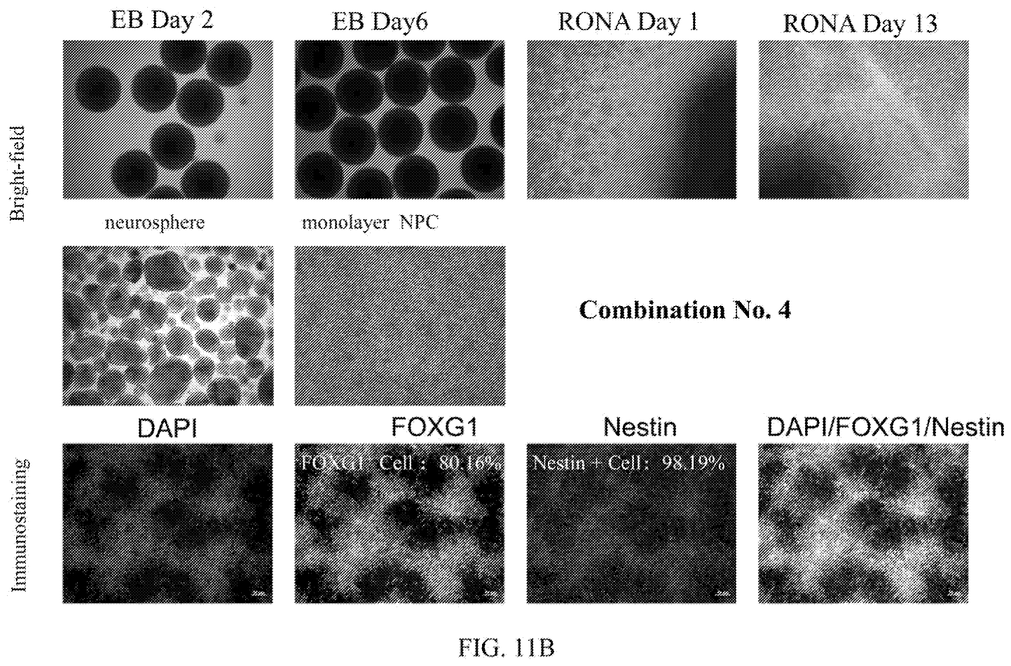

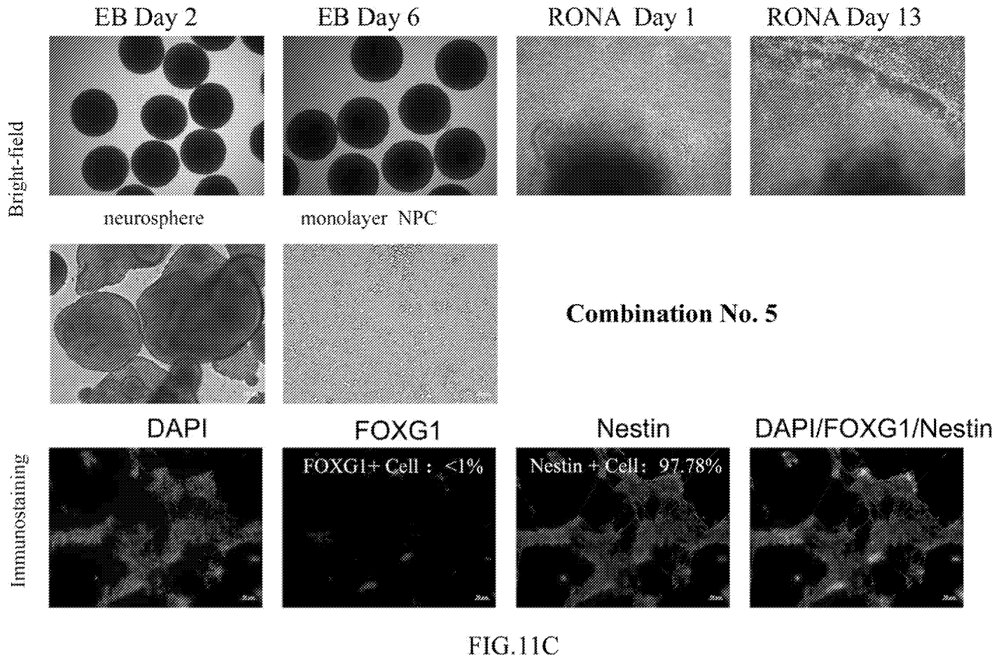

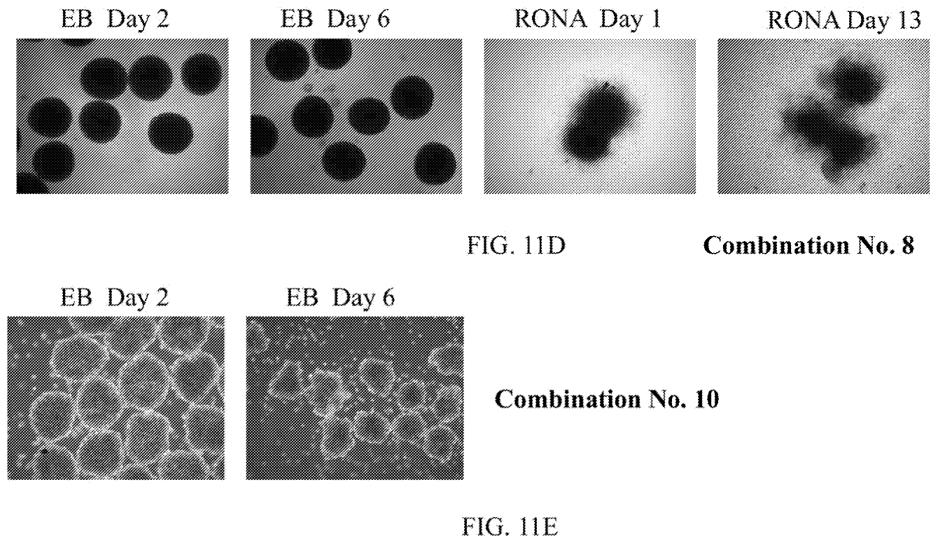

TABLE 3
Detailed testing results of different EB and NIM compositions

| | | | Immunostaining results | |
|---|---|---|---|---|
| No. | Code name | Descriptive results | FOXG1⁺ cells | Nestin⁺ cells |
| 1 | CTS-NDM + NDS/CTS-NDM | Successful formation of NPCs | 99.54% | 99.58% |
| | | (FIG. 11A) | | |
| 2 | CTS-N2M + NDS/CTS-N2M | Successful formation of NPCs | 99.67% | 99.87% |
| 3 | CTS-N2B27M + NDS/CTS- | Successful formation of NPCs | 96.99% | 98.56% |
| | N2B27M | | | |
| 4 | GMP-N2M + NDS/GMP-N2M | Successful formation of NPCs | 80.16% | 98.16% |
| | | (FIG. 11B) | | |
| 5 | GMP-N2B27M + NDS/GMP- | Successful formation of NPCs. But a | <1% | >90% |
| | N2B27M | substantial number of dead cells could be | | |
| | | observed along with the formation of | | |
| | | monolayer NPCs (FIG. 11C) | | |
| 6 | GMP-EBM + NDS/GMP-NIM | Successful formation of NPCs. But a | <3% | >95% |
| | | substantial number of dead cells could be | | |
| | | observed along with the formation of | | |
| | | monolayer NPCs | | |
| 7 | GMP-EBM + NDS/GMP-NIM | Successful formation of NPCs. But a | <5% | >95% |
| | | substantial number of dead cells could be | | |
| | | observed along with the formation of | | |
| | | monolayer NPCs | | |
| 8 | BI-EB + NDS/CTS-NIM | Experiment was discontinued as it failed to | N/A | N/A |
| | | form rosette-like aggregates (RONAs) (FIG. | | |
| | | 11D) | | |
| 9 | CTS-NIM + NDS/CTS-NIM | Experiment was discontinued as it failed to | N/A | N/A |
| | | harvest healthy spherical EBs after initial | | |
| | | differentiation | | |
| 10 | CTS-EBM + NDS/CTS-NIM | Experiment was discontinued as it failed to | N/A | N/A |
| | | harvest healthy spherical EBs after initial | | |
| | | differentiation (FIG. 11E) | | |

It can be seen from the table above and FIGS. 11A-E, test combinations 1-4 which represent the medium compositions of the present application provided excellent or fairly good results.

### Example 8. Directed Differentiation Started with Stem Cell Colonies

In the preferred embodiment of present application, for example the method as described in Example 1 or Example 4, EBs were formed by digesting the stem cells and seeding the cells at a certain density in a culture plate. In this example, the present inventors tested a method started with stem cell colonies instead of a predetermined number of single stem cells. This example mirrors the procedure previously published in Xu et al., 2016, while the medium and supplements were replaced by their clinical grade counterparts.

ESC or iPSC were maintained in plates coated with CTS™ Vitronectin in NutriStem® hPSC XF medium. The ESC or iPSC colonies were treated with Collagenase NB 6 GMP Grade (0.15 PZ U/mL HBSS, Calcium, Magnesium, no Phenol Red) for about 20 min. Without counting cell number, the detached colonies were directed transferred to and grown in CTS-EBM for one day. The CTS-EBM was prepared by adding 20% KnockOut™ SR XenoFree CTS™ (Gibco), 1 vol % GMP grade MEM Non-Essential Amino Acids Solution (Gibco), 0.5 vol % CTS™ GlutaMAX™-I Supplement (Gibco) into CTS™ KnockOut™ DMEM/F-12.

The rest of the procedure is identical to the steps in Example 1 and Example 4. From day 2-6, the EB medium used to culture the cells was CTS-EBM+NDS. CTS-EBM+NDS containing 50 ng/mL Noggin GMP, 1 μM Dorsomorphin, 10 μM SB431542, 20% KnockOut™ SR XenoFree CTS™ (Gibco), 1 vol % GMP grade MEM Non-Essential Amino Acids Solution (Gibco), 0.5 vol % CTS™ GlutaMAX™-I Supplement (Gibco) and CTS™ KnockOut™ DMEM/F-12.

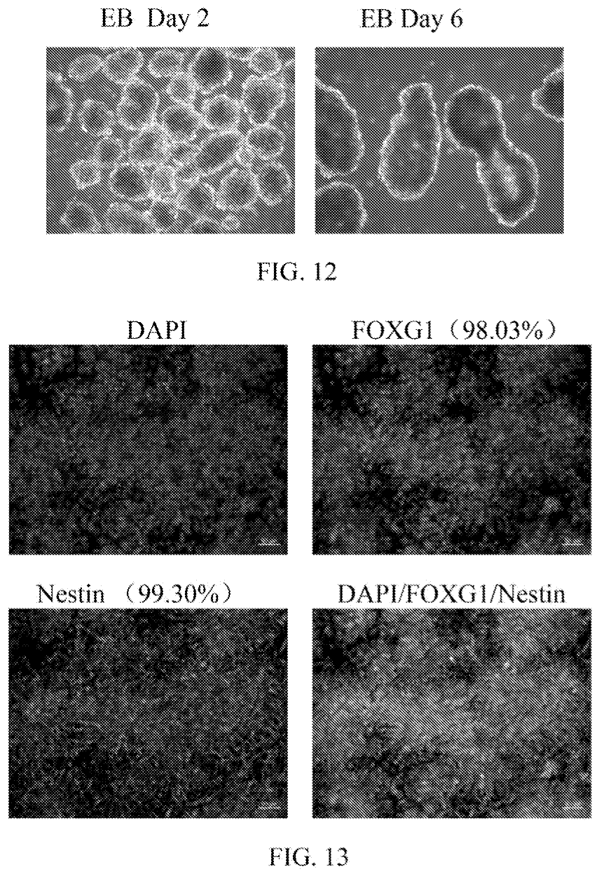

The EBs formed at Day 6 did not show a sphere-like shape (FIG. 12), which failed to differentiate into NPCs. The results showed the uncertainty in translating a protocol proven to be successful in laboratory to clinics. Simply replacing the basal media and supplements with their clinical-grade counterparts cannot guarantee successful generation of clinical grade NPCs.

### Example 9. Directed Differentiation by Using a Combination of Two NIMs

The same process as described in Example 1 or Example 4 was performed except for successively using two NIMs to induce neural differentiation. On day 7 after initial differentiation, EBs were transferred into 6 well plates coated with clinical-grade Laminin-521 (BioLamina, MX521/CT521) to allow complete attachment of EBs, and cultured with two different types of clinical-grade serum-free neural induction medium (NIM) which promotes the expansion of the neural stem cells and NPCs. From Day 1 to Day 7 during the differentiation of RONAs, a clinical-grade serum-free N2M was used, which was prepared by mixing CTS™ KnockOut™ DMEM/F-12 Medium (Gibco) and CTS™ Neurobasal™ Medium (Gibco) at ratio of 1:1 (vol/vol) to obtain a basal medium, and adding the basal medium with 1 vol % of CTS™ N-2 Supplement (100×) (Gibco), 0.5 vol % of CTS™ GlutaMAX™-I Supplement (200×) (Gibco). From Day 8 to Day 14 during the differentiation of RONAs, a clinical-grade serum-free NDM was used, which was prepared by mixing CTS™ KnockOut™ DMEM/F-12 Medium (Gibco) and CTS™ Neurobasal™ Medium (Gibco) at ratio of 1:1 (vol/vol) to obtain a basal medium, and adding the basal medium with 0.5 vol % of CTS™ N-2 Supplement (200×) (Gibco), 1 vol % CTS™ grade B-27™ Supplement, XenoFree, minus vitamin A (100×) (Gibco) and 0.5 vol % of CTS™ GlutaMAX™-I Supplement (200×) (Gibco).

Immunofluorescent staining assay was performed on the hNPCs obtained by the method as described in this Example. As shown by FIG. 13, most of the cells showed expressions of both FOXG1 and Nestin, indicating successful generation of a bulk of hNPCs with high purity by differentiation. Specifically, among the population of generated cells, a proportion of FOXG1⁺ cells as high as 98.03%, and a proportion of Nestin⁺ cells as high as 99.30% were achieved (FIG. 13).

Immunofluorescent staining assays were performed to show the in vitro differentiation of the hNPCs obtained from the expansion culture in Example 9 of the present invention.

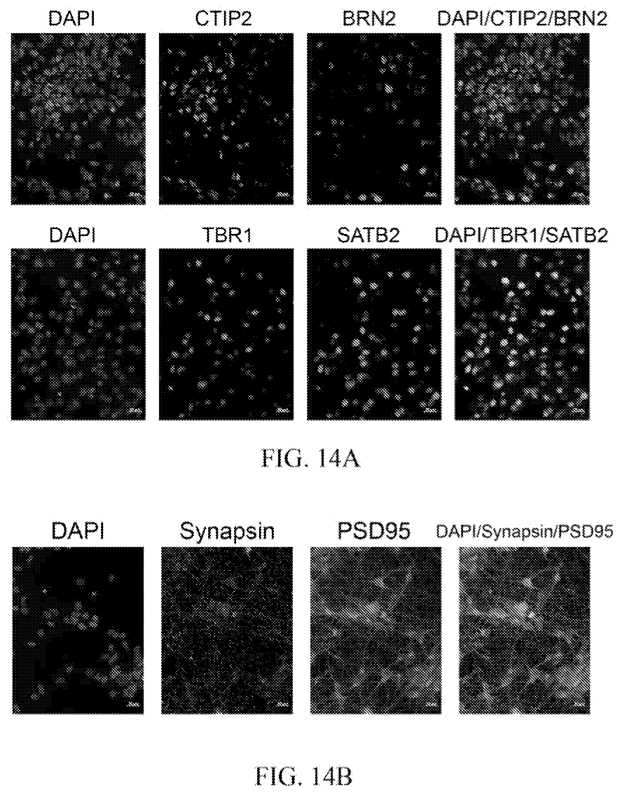

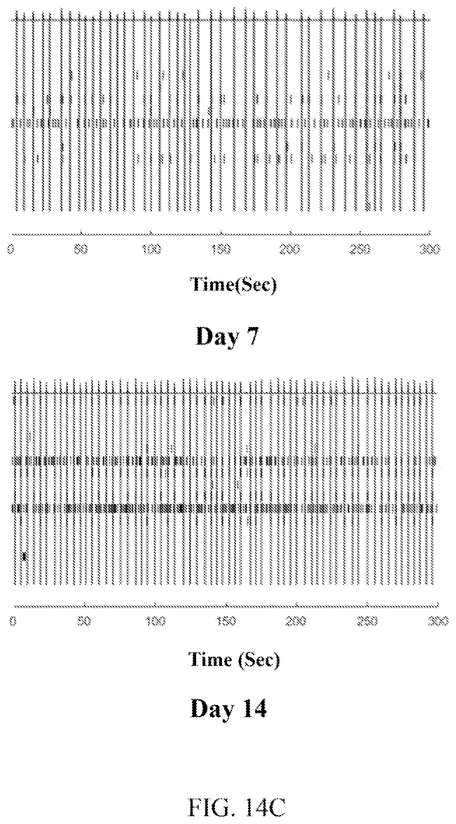

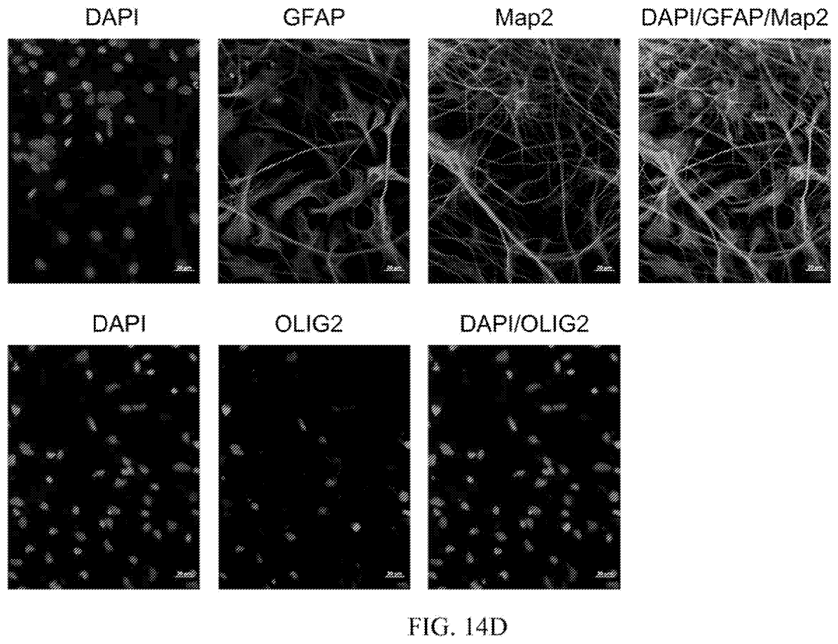

FIG. 14 A shows the results of immunofluorescent staining performed after 2 months of in vitro differentiation of hNPCs, which detected the expression of the markers specific for the six layers of human cerebral cortex. As shown by FIG. 14A, the fluorescent signals represented expression of the markers, which were BRN2 and SATB2 for layers II to IV, CTIP2 for layers V and VI, TBR1 for layers I, V, and VI. The results of the immunofluorescent staining demonstrated that the hNPCs obtained by the method of Example 9 had successfully differentiated into nerve cells of all six layers of human cerebral cortex.

FIG. 14B shows the results of immunofluorescent staining for Synapsin and PSD95, indicating the expression of these two markers which are presynaptic marker and postsynaptic marker, respectively. As shown by FIG. 14B, the signals representing expression of synaptic protein were distributed as discrete points, indicating maturation of neurons after differentiation. As shown by the FIG. 14C, electrophysiological activities could be detected on Day 7 of in vitro differentiation. Moreover, as the culture time was extended, spike firing gradually intensified, and more regular spontaneous firing and network bursts were observed.

After 3 months of in vitro differentiation of hNPCs obtained by the method as described in Example 9, immunofluorescent staining assays were performed to detect the expression of Map2, GFAP and OLIG2. As shown by FIG. 14D, the positive signals of Map2, GFAP and OLIG2 indicated that neurons, astrocytes and oligodendrocyte progenitor cells were further differentiated from the hNPCs obtained by the method of the present application at this time point.

Taken together, the above-mentioned immunostaining assays demonstrated that the hNPCs generated from iPSCs by the method as described in Example 9 are capable of differentiating into cells of all six layers of human cerebral cortex with desirable physiological functions.

1. A method of generating neural progenitor cells (NPCs) from embryonic stem cells (ESCs) or induced pluripotent stem cells (iPSCs), including:
(1) culturing ESCs or iPSCs in human pluripotent stem cell medium (hPSC medium) to allow formation of embryoid bodies (EBs);
(2) culturing the EBs formed by step (1) in EB medium comprising a basal medium and supplements;
(3) culturing the EBs after step (2) on an extracellular matrix (ECM)-coated culture surface with neural induction medium (NIM) to form ROsetteNeural Aggregates (RONAs), wherein the NIM comprises a basal medium and supplements;
(4) culturing the RONAs formed by step (3) to form neurospheres; and
(5) disassociating neurospheres into single cells and culturing on an ECM-coated culture surface with NPC medium to form monolayer NPCs, wherein the NPC medium comprises a basal medium and supplements;
wherein the hPSC medium, and the basal media and supplements comprised in the EB medium, the NIM, and the NPC medium are clinical grade.

2. The method of claim 1, one or more, preferably all of the basal media and supplements, comprised in the EB medium, the NIM, and the NPC medium, are GMP grade, cGMP grade or CTS™ grade.

3. The method of claim 1, wherein one or more, preferably all of the hPSC medium, the EB medium, the NIM, and the NPC medium are clinical grade, preferably GMP grade, cGMP grade or CTS™ grade.

4. The method of claim 1, wherein the hPSC medium is NutriStem® hPSC XF Medium, CTS™ Essential 8 Medium, StemFit® Basic03, StemMACS™ iPS-Brew, Stem-Partner® ACF, TeSR™-AOF and TeSR2.

5. The method of claim 1, wherein the hPSC medium is supplemented with ROCK inhibitors.

6. The method of claim 1, wherein the EB medium comprises
(i) a basal medium selected from a group consisting of a) to c)
a) KnockOut™ DMEM/F12 medium alone,
b) DMEM/F12 in combination with Neurobasal™ Medium,
c) KnockOut™ DMEM/F12 medium in combination with Neurobasal™ Medium; and
(ii) supplements comprising or consisting of d) or e)
d) N-2 Supplement and GlutaMAX™-I Supplement;
e) N-2 Supplement, GlutaMAX™-I Supplement and B-27™ Supplement, minus vitamin A.

7. The method of claim 6, wherein the EB medium further comprises inhibitors, wherein the inhibitors comprise or consist of a BMP inhibitor, an AMPK inhibitor and an ALK inhibitor, preferably comprise or consist of one or more of Noggin, SB431542, LDN-193189, DMH-1 and Dorsomorphin, preferably SB431542 in combination with any one or more, for example one or two of Noggin, LDN-193189, DMH-1 and Dorsomorphin. e.g. SB431542 in combination with one or two of Noggin, LDN-193189, DMH-1 and Dorsomorphin.

8. The method of claim 5, wherein the ROCK inhibitor is clinical grade, preferably GMP grade, cGMP grade or CTS™ grade.

9. The method of claim 1, wherein the NIM comprises
(i) a basal medium selected from a group consisting of a) to c)
a) KnockOut™ DMEM/F12 medium alone,
b) DMEM/F12 in combination with Neurobasal™ Medium,
c) KnockOut™ DMEM/F12 medium in combination with Neurobasal™ Medium; and
(ii) supplements comprising or consisting of d) or e)
d) N-2 Supplement and GlutaMAX™-I Supplement;
e) N-2 Supplement, GlutaMAX™-I Supplement and B-27™ Supplement, minus vitamin A.

10. The method of claim 1, wherein the culturing of step (4) is conducted by using NIM or NPC medium.

11. The method of claim 1, wherein the basal medium of NPC medium is Neurobasal™ Medium, preferably CTS™ Neurobasal™ Medium, and the supplements of NPC medium are (a) GlutaMAX™-1 Supplement, preferably CTS™ GlutaMAX™-I Supplement, and (b) B-27™ Supplement, minus vitamin A, preferably cGMP grade or CTS™ grade B-27™ Supplement, XenoFree, minus vitamin A.

12. The method of claim 11, wherein the NPC medium further comprises brain-derived neurotrophic factor (BDNF), and/or the glial cell line-derived neurotrophic factor (GDNF), and/or L-ascorbic acid, and/or N⁶, O²′-Dibutyryl Adenosine 3′,5′ cyclic-monophosphate sodium salt (DB-cAMP).

13. The method of claim 12, wherein the BDNF is Animal-Free Recombinant BDNF or GMP grade Recombinant BDNF, and/or the GDNF is Animal-Free Recombinant GDNF or GMP grade Recombinant GDNF.

14. The method of claim 1, wherein the ESCs or iPSCs in step (1) is dispersed into single cells or cell aggregates e.g. by a digestion enzyme or by mechanical means before being seeded into the hPSC medium to induce formation of EBs.

15. The method of claim 1, wherein more than one EB medium is used in step (2), and/or more than one NIM is used in step (3).

16. NPCs generated by the method of claim 1 or cells derived therefrom.

17. The NPCs or cells derived therefrom according to claim 16, which are suitable for use in drug development, pre-clinical use or clinical use.

18. (canceled)

19. A method of generating neurons, astrocyte progenitor cells, astrocytes, oligodendrocyte progenitor cells, oligodendrocytes, or mixed cell population comprising one or more of those cells from the NPCs generated by the method of claim 1, preferably by using medium of clinical grade, preferably GMP grade, cGMP grade or CTS™ grade.

20. (canceled)

21. The method of claim 7, wherein the one or more of the inhibitors are clinical grade, preferably GMP grade, cGMP grade or CTS™ grade.
